(19) United States
(12) Patent Application Publication
(10) Pub. No.: US 2007/0110758 A1
(43) Pub. Date: May 17, 2007

(54) Method for inhibiting bacterial colonisation

(75) Inventors: Fiona Campbell, South Australia (AU); Ross Butler, South Australia (AU); Tran Cuong, South Australia (AU); Hien Huynh, South Australia (AU); Richard Couper, South Australia (AU)

Correspondence Address: THE MCCALLUM LAW FIRM, P. C., 685 BRIGGS STREET, PO BOX 929, ERIE, CO, 80516, US

(73) Assignee: ADELAIDE RESERCH & INNOVATION PTY LTD, ADELAIDE SOUTH AUSTRIALIA (AU)

(21) Appl. No.: 10/567,659

(22) PCT Filed: Aug. 9, 2004
(86) PCT No.: PCT/AU04/01059

§ 371 Date: Sep. 7, 2006

(30) Foreign Application Priority Data
Aug. 8, 2003 (AU) 2003904192

(51) Int. Cl. A61K 39/00; A61K 38/40; A61K 31/192; A61K 35/20; A61K 31/43

(52) U.S. Cl. 424/184.1; 424/535; 514/562; 514/6; 514/192

(57) ABSTRACT

The present invention relates to a method for inhibiting bacterial colonisation of mucous epithelium in a biological system. The method includes the step of administering to the biological system an effective amount of a mucolytic agent and one or more of colostrum, hyperimmune milk, or a component of colostrum and/or hyperimmune milk that is capable of inhibiting bacterial colonisation in combination with the mucolytic agent.

### FIELD OF THE INVENTION

The present invention relates to methods and compositions for inhibiting the bacterial colonisation of mucous epithelium in biological systems, and also to methods and compositions for reducing infection, inflammation and damage to mucous epithelium caused by bacterial colonisation of mucous epithelium.

### BACKGROUND OF THE INVENTION

Many diseases and conditions are associated with the colonisation and infection of mucosal surfaces by pathogenic bacteria. The mucosal surface of organs and tissues such as the gastrointestinal tract, the oral cavity, the respiratory tract, oesophagus, mouth, genitourinary tract, and eye may all be colonised and infected by numerous different types of pathogenic bacteria. For example, the colonisation and infection of the gastric mucosa by Helicobacter pylori plays a key role in the development of a number of clinical manifestations, including gastritis, gastric and duodenal ulcers, gastric adenocarcinoma, mucosa-associated lymphoid tissue lymphoma and non-ulcer dyspepsia.

The ability of bacteria to colonise and infect such mucosal surfaces involves many factors, including the ability of the pathogenic bacteria to adhere to host cells and resist physical removal, the ability of the bacteria to invade host cells, the ability of the bacteria to resist phagocytosis and complement, the ability of the bacteria to evade host immune defences, and the ability to compete with host tissue and normal flora for limited nutrients.

For example, Neisseria gonorrhea synthesizes different pili that allow it to adhere to mucosal surfaces of a variety of tissues, including the throat, genitourinary tract, rectum and conjunctiva of the eye. Streptococcus pyogenes produces adhesions, proteins that bind to a specific receptor on the surface of host cells. Some bacteria, such as Shigella strains, produce molecules that activate the cytoskeletal machinery of the host cell enabling bacterial entry into the cell by phagocytosis.

In the case of infection of the gastric mucosa by H. pylori, the bacterium utilises a number of different mechanisms to colonise and infect the stomach beneath the gastric mucosa. H. pylori first colonises the antrum of the stomach, due to the moderate acidity of this region. The bacterium then uses its flagella and spiral shape to drill through the gastric mucous layer. Adhesins produced then allow binding to membrane-associated lipids and carbohydrates of epithelial cells. Finally, the bacterium produces the enzyme urease, which facilitates colonisation of the acidic gastric environment. The urease digests urea to produce ammonia and bicarbonate, aiding in the neutralization of gastric acid. The weakening of the stomach's protective mucous layer makes the stomach susceptible to the damaging effects of acid and pepsin.

The presence of H. pylori in the gastric mucosa will invariably be associated with mucosal inflammation due to infiltration by neutrophils and monocytes. A number of harmful enzymes are also produced by H. pylori and these are also likely to be involved in inflammation of the gastric mucosa. The inflammation of the gastric mucosa may also lead to further damage to the stomach.

The treatments for eradication of bacteria that colonise and infect mucosa are generally expensive, lack efficacy and are only advisable under certain clinical conditions. Many treatment regimes are also often complicated, produce serious side effects and are difficult for the patient to comply with. The treatment regimes often involve multiple agents, including one or more antibiotics. For example, the current recommended treatment for eradication of H. pylori involves a triple therapy regime using antibiotics and a proton pump inhibitor. It is also unclear if vaccination will ever be a realistic and effective prophylactic treatment of diseases and conditions associated with the colonisation and infection of mucosa by a number of pathogenic bacteria.

Accordingly, there is a need for new methods and compositions that inhibit the colonisation and infection of mucous epithelium by bacteria.

The present invention relates to the identification of a combination of agents that act to inhibit the colonisation, infection and associated inflammation of mucous epithelium by bacteria.

Throughout this specification reference may be made to documents for the purpose of describing various aspects of the invention. However, no admission is made that any reference cited in this specification constitutes prior art. In particular, it will be understood that the reference to any document herein does not constitute an admission that any of these documents forms part of the common general knowledge in the art in any other country. The discussion of the references states what their authors assert, and the applicant reserves the right to challenge the accuracy and pertinency of any of the documents cited herein.

### SUMMARY OF THE INVENTION

The present invention provides a method for inhibiting bacterial colonisation of mucous epithelium in a biological system, the method including the step of administering to the biological system an effective amount of a mucolytic agent and one or more of colostrum, hyperimmune milk, or a component of colostrum and/or hyperimmune milk that is capable of inhibiting bacterial colonisation in combination with the mucolytic agent.

The present invention also provides a method for reducing bacterial infection of mucous epithelium in a biological system, the method including the step of administering to the biological system an effective amount of a mucolytic agent and one or more of colostrum, hyperimmune milk, or a component of colostrum and/or hyperimmune milk that is capable of reducing bacterial infection in combination with the mucolytic agent.

The present invention further provides a method for reducing inflammation associated with bacterial infection of mucous epithelium in a biological system, the method including the step of administering to the biological system an effective amount of a mucolytic agent and one or more of colostrum, hyperimmune milk, or a component of colostrum and/or hyperimmune milk that is capable of reducing inflammation associated with bacterial infection in combination with the mucolytic agent.

The present invention also provides a method for reducing damage to mucous epithelium associated with bacterial infection of the mucous epithelium in a biological system, the method including the step of to the biological system an effective amount of a mucolytic agent and one or more of colostrum, hyperimmune milk, or a component of colostrum and/or hyperimmune milk that is capable of reducing the damage to mucous epithelium associated with bacterial infection in combination with the mucolytic agent.

The present invention also provides a method for treating a disease or condition associated with bacterial infection of mucous epithelium in a subject, the method including the step of administering to the subject an effective amount of a mucolytic agent and one or more of colostrum, hyperimmune milk, or a component of colostrum and/or hyperimmune milk that is capable of treating the disease or condition associated with bacterial infection of mucous epithelium in the subject in combination with the mucolytic agent.

The present invention further provides a composition including a mucolytic agent and one or more of colostrum, hyperimmune milk, or a component of colostrum and/or hyperimmune milk.

The present invention arises out of studies into the ability of colostrum to inhibit colonisation and infection of mucous epithelium by bacteria that are associated with diseases or conditions of the mucous epithelium. It has been surprisingly found that the capacity to inhibit bacterial colonisation, infection and the associated inflammation of the stomach is improved by a combination of colostrum (or a component of colostrum) and a mucolytic agent. In particular, it has been found that colonisation, infection and the associated inflammation of the stomach by H. pylori may be inhibited or prevented by treatment with colostrum, or a component of colostrum, in combination with a mucolytic agent such as N-acetyl cysteine.

Various terms that will be used throughout the specification have meanings that will be well understood by a skilled addressee. However, for ease of reference, some of these terms will now be defined.

The term “mucous epithelium” as used throughout the specification is to be understood to mean any collection of epithelial cells that contain cells that secrete mucous and produce a layer of mucous able to be colonised by bacteria.

The term “biological system” as used throughout the specification is to be understood to mean any multi-cellular system having mucous epithelium. For example, the biological system may be the whole or part of an organ or tissue having mucous epithelium, or an entire animal or human subject susceptible to or suffering the effects of colonisation or infection of mucous epithelium by bacteria.

The term “mucolytic agent” as used throughout the specification is to be understood to mean any agent that has the capacity to reduce the hydrophobicity of mucous. A validated technique for the measurement of hydrophobicity is the measurement of contact angles of biopsy specimens when a drop of saline is placed upon the surface of the specimen, as described in Absolom et al. (1986) J. Colloid Interface Sci 112:599. Contact angles may be measured using a goniometer fitted with a monochromatic light source and micrometer-activated syringe for applying small volumes of saline to the tissue surface. A small volume of saline (5 μL) may be applied to the surface of the tissue. The centre of the field of view may be adjusted to coincide with the triple point, and then one cross hair may be adjusted to coincide with the tissue-fluid interface. The angle between the two is the contact angle and this may be read directly from the scale encircling the eyepiece.

The phrase “colonisation of mucous epithelium” as used throughout the specification is to be understood to mean the establishment of one or more bacteria beneath and/or within a layer of mucous associated with mucous epithelium.

The terms “reduce” and “inhibit” as used throughout the specification are to be understood to mean a reduction or inhibition of the progress of a process, including the start, continuation or termination of a process, and in the context of the present invention these terms include the prevention of bacterial colonisation, infection and inflammation of mucous epithelium by bacteria.

The phrase “infection of mucous epithelium” as used throughout the specification is to be understood to mean the presence of one or more bacteria beneath and/or within layer of mucous associated with mucous epithelium.

The phrase “damage to mucous epithelium” as used throughout the specification is to be understood to mean the damage to mucous epithelium that occurs as a result of infection of the mucous epithelium by bacteria. Such damage can be damage that results directly from the colonisation or infection by bacteria, and/or be damage that results indirectly from the infection of mucous epithelium by bacteria, such as the damage that occurs as a result of the inflammation of the mucous epithelium.

The phrase “anti-bacterial agent derived from a milk product” as used throughout the specification is to be understood to mean any component of milk, hyperimmune milk, colostrum, hyperimmune colostrum or any other milk derived product that has anti-bacterial activity (either bactericidal or bacteriostatic) produced by a method known in the art. This includes one or more fractions or extracts derived from milk, hyperimmune milk, colostrum or hyperimmune colostrum, or any component with anti-bacterial activity in a composition that would normally be present in milk, hyperimmune milk, colostrum or hyperimmune colostrum, including substantially purified products from milk, hyperimmune milk, colostrum or hyperimmune colostrum, or a product produced by recombinant DNA technology.

### BRIEF DESCRIPTION OF THE FIGURES

FIG. 1 shows in panel A the level of colonisation in the gastric body of mice treated with saline (NaCl), hyperimmune colostrum (HBC) or bovine lactoferrin (BLf). Panel B shows the level of colonisation in the antrum and panel C shows the level of comonisation in the stomach overall. The results for individual animals are represented by ♦ symbols; the mean response in each group is indicated by a horizontal bar (—) and the numerical value.

FIG. 2 shows in panel A the overall level of colonisation in mice treated with water (H₂O), bovine lactoferrin (BLf), or bovine lactoferrin with N-acetyl cysteine (BLf*). Results for individual animals are represented by □ symbols; the mean response in each group is indicated by a horizontal bar (—) and the numerical value. Panel B shows the level of colonisation in the transitional zone.

FIG. 3 shows in panel A the level of colonisation in the gastric body of mice treated with water alone (H₂O), N-acetyl cysteine alone (NAC), bovine lactoferrin pepsin hydrolysate (BLc-A), bovine lactoferrin acid hydrolysate (BLc-B), non-immune bovine colostrum and N-acetyl cysteine (NBC*), hyperimmune bovine colostrum and N-acetyl cysteine (HBC*), bovine lactoferrin and N-acetyl cysteine (BLf*), bovine lactoferrin pepsin hydrolysate and N-acetyl cysteine (BLc-A*), bovine lactoferrin acid hydrolysate and N-acetyl cysteine (BLc-B*), or in mice treated with triple therapy regimen (TT). Panel B shows the level of colonisation in the transitional zone. Results for individual animals are represented by — symbols; the mean response in each group is indicated by a horizontal bar (—) and the numerical value.

FIG. 4 shows in panel A the level of colonisation in the antrum of mice treated with water alone (H₂O), N-acetyl cysteine alone (NAC), bovine lactoferrin pepsin hydrolysate (BLc-A), bovine lactoferrin acid hydrolysate (BLc-B), non-immune bovine colostrum and N-acetyl cysteine (NBC*), hyperimmune bovine colostrum and N-acetyl cysteine (HBC*), bovine lactoferrin and N-acetyl cysteine (BLf*), bovine lactoferrin pepsin hydrolysate and N-acetyl cysteine (BLc-A*), bovine lactoferrin acid hydrolysate and N-acetyl cysteine (BLc-B*), or in mice treated with triple therapy regimen (TT). Panel B shows the level of colonisation in the mouse stomach when considered overall. Results for individual animals are represented by — symbols; the mean response in each group is indicated by a horizontal bar (—) and the numerical value.

FIG. 5 shows the overall level of chronic inflammatory cell infiltration (chronic gastritis) in mice treated with water (H₂O), bovine lactoferrin (BLf, or bovine lactoferrin in combination with N-acetyl cysteine (BLf*). Results for individual animals are represented by ♦ symbols, and the mean response in each group is indicated by a horizontal bar (—) and the numerical value.

FIG. 6 shows in panel A the level of inflammatory cell infiltration in the gastric body of mice treated with water alone (H₂O), hyperimmune bovine colostrum and N-acetyl cysteine (HBC), bovine lactoferrin and N-acetyl cysteine (BLf), bovine lactoferrin pepsin hydrolysate and N-acetyl cysteine (BLc-A), or bovine lactoferrin acid hydrolysate and N-acetyl cysteine (BLc-B). Panel B shows the level of inflammatory cell infiltration in the transitional zone of mice. For panel A and B, results for individual animals are represented by symbols (X or □), and the mean response in each group is indicated by a horizontal bar (—) and the numerical value. Panel C shows the level of inflammatory cell infiltration in the gastric antrum of mice. Panel D shows the combined score of inflammatory cell infiltration. For panel C and D, results for individual animals are represented by symbols (◯ or ♦); the mean response in each group is indicated by a horizontal bar (—) and the numerical value.

FIG. 7 shows the level of acute gastritis (MPO activity) detected in mice treated with either bovine lactoferrin (BLf) or bovine lactoferrin in combination with N-acetyl cysteine (BLf*) was compared to the level of acute gastritis (MPO activity) in H₂O control mice. Results for individual animals are represented by symbols; the mean response in each group is indicated by a horizontal bar (—) and the numerical value.

FIG. 8 shows the level of acute gastritis (MPO activity) in mice treated with water alone (H₂O), hyperimmune bovine colostrum and N-acetyl cysteine (HBC), bovine lactoferrin and N-acetyl cysteine (BLf), bovine lactoferrin pepsin hydrolysate and N-acetyl cysteine (BLc-A), or bovine lactoferrin acid hydrolysate and N-acetyl cysteine (BLc-B). Results for individual animals are represented by symbols (□) whereas the mean response in each group is indicated by a horizontal bar (—) and the numerical value.

FIG. 9 shows in panel A the viable count of SS1 colonisation according to the various treatment regimes as described in Example 11, and in panel B the MPO activity in gastric tissue according to the various treatment regimes as described in Example 11.

### GENERAL DESCRIPTION OF THE INVENTION

As mentioned above, in one form the present invention provides a method for inhibiting bacterial colonisation of mucous epithelium in a biological system, the method including the step of administering to the biological system an effective amount of a mucolytic agent and one or more of colostrum, hyperimmune milk, or a component of colostrum and/or hyperimmune milk that is capable of inhibiting bacterial colonisation in combination with the mucolytic agent.

The mucous epithelium according to the various forms of the present invention may be any mucous epithelium that has become colonised or infected by bacteria, or any mucous epithelium that has the capacity to become colonised or infected by bacteria. Preferably, the mucous epithelium is mucous epithelium of an animal or human. Most preferably, the mucous epithelium is mucous epithelium of a human.

The mucous epithelium may be part of one or more of the following organs or tissues: stomach, including the cardia, fundus, body, antrum and pylorus of the stomach; duodenum; ileum; small intestine; large intestine; colon; bowel; rectum; esophagus; mouth; tongue; pharynx; urino-genital tact; eye; and respiratory tract, including the nasal cavity, oral cavity, larynx, trachea, bronchi including bronchioles and alveoli, and lungs. Preferably, the mucous epithelium is mucous epithelium of one or more of the cardia, fundus, antrum or pylorus of the stomach.

The mucous epithelium may be any mucous epithelium associated with a disease or condition associated with the colonisation or infection of mucous epithelium by bacteria. In this regard, the diseases or conditions associated with the colonisation or infection of mucous epithelium by bacteria include gastric inflammation; ulcers of the stomach or duodenum; gastric adenocarcinoma; mucosa-associated lymphoid tissue lymphoma; non-ulcer dyspepsia; gastric conditions associated with leukocyte infiltration; urinary tract infections; strep throat; infective endocarditis; bacterial pneumonia; whooping cough; gingivitis; acute or chronic bronchitis; bronchiectasis; asthmatic bronchitis; bronchial asthma; bronchiolitis; cystic fibrosis; laryngopharyngitis; acute or chronic rhinitis. Preferably, the mucous epithelium is mucous epithelium associated with gastric inflammation, ulcers of the stomach or duodenum, gastric adenocarcinoma, mucosa-associated lymphoid tissue lymphoma, non-ulcer dyspepsia, or a gastric condition associated with leukocyte infiltration.

The bacteria that may colonise or infect mucous epithelium according to the various forms of the present invention include Helicobacter species including Helicobacter pylori, Helicobacter hepaticus, Helicobacter rappini, Helicobacter muridarum, Helicobacter bilis; Streptococcus species including Streptococcus mutans, Streptococcus pyogenes, Streptococcus pnuemoniae; Enterococci species including Enterococcus faecalis; Bacteroides species; Bifidobacterium species; Peptococcus species; Peptostreptococcus species; Ruminococcus species; Clostridia species including Clostridium difficile; Lactobacillus species including Lactobacillus acidophilus; Neisseria species including Neisseda gonorrhea, Neisseria meningitides; Escherichia coli; Vibrio cholerae; Shigella species including Shigella dysenteriae, Shigella flexneni, and Shigella Sonnei, Yersinia species including Yersinia enterocoliftica; Pseudomonas aeruginosa; Bordetella pertuśsis; Campylobacter species including Campylobacter jejuni; Haemophilus influenzae; Staphyloccus species including Staphyloccus epidermis, Staphyloccus aureus. Preferably, the bacteria that may colonise or infect mucous epithelium is a bacteria of the Helicobacter species. Most preferably, the bacteria is Helicobacter pylori.

The biological system according to the various forms of the present invention may be any multi-cellular system having mucous epithelium, including the whole or part of an organ or tissue, or an entire human or animal subject, and which includes mucous epithelium that has the capacity to be colonised or infected by bacteria.

Preferably, the biological system is a human or animal. More preferably, the biological system is a human or animal with mucous epithelium associated with a disease or condition resulting from the colonisation or infection by bacteria. Most preferably, the biological system is a human subject susceptible to, or actually suffering from, one or more of the following diseases or conditions due to the colonisation or infection of mucous epithelium by bacteria: gastric inflammation; ulcers of the stomach or duodenum; gastric adenocarcinoma; mucosa-associated lymphoid tissue lymphoma; non-ulcer dyspepsia; gastric conditions associated with leukocyte infiltration; urinary tract infections; strep throat; infective endocarditis; bacterial pneumonia; whooping cough; gingivitis; acute or chronic bronchitis; bronchiectasis; asthmatic bronchitis; bronchial asthma; bronchiolitis; cystic fibrosis; laryngopharyngitis; acute or chronic rhinitis.

The mucolytic agent according to the various forms of the present invention is an agent that has the capacity to reduce the hydrophobicity of mucous. A validated technique for the measurement of hydrophobicity is the measurement of contact angles of biopsy specimens when a drop of saline is placed upon the surface of the specimen, as described in Absolom et al. (1986) J. Colloid Interface Sci 112:599. Contact angles may be measured using a goniometer fitted with a monochromatic light source and micrometer-activated syringe for applying small volumes of saline to the tissue surface. A small volume of saline (5 μL) may be applied to the surface of the tissue. The centre of the field of view may be adjusted to coincide with the triple point, and then one cross hair may be adjusted to coincide with the tissue-fluid interface. The angle between the two is the contact angle and this may be read directly from the scale encircling the eyepiece.

Examples of mucolytic agents include N-acetyl cysteine, t-butyl cysteine, fatty acid derivatives of cysteine, N-guanyl-cysteine, ethylcysteine, nesosteine, ambroxol, Dnase, iodine, Gesolin, sodium 2-mercaptoethanesulphonate, carbocisteine and mecysteine, and bromhexine. Preferably, the mucolytic agent is N-acetyl cysteine.

Accordingly, in a preferred form the present invention provides a method for inhibiting bacterial colonisation of mucous epithelium in a biological system, the method including the step of administering to the biological system an effective amount of a N-acetyl cysteine and one or more of colostrum, hyperimmune milk, or a component of colostrum and/or hyperimmune milk that is capable of inhibiting bacterial colonisation in combination with N-acetyl cysteine.

The colostrum according to the forms of the present invention may be any colostrum that is secreted by a mammal, including human colostrum, bovine colostrum, ovine colostrum, caprine colostrum, porcine colostrum, or equine colostrum. Preferably, the colostrum is bovine colostrum.

The colostrum may be collected by a suitable method known in the art, such as that described in Davidson et al. (1989) Lancet 2: 709-712.

Preferably, the colostrum is hyperimmune colostrum resulting from the successive immunization of a mammal with the bacteria (or antigens derived from the bacteria) for which colonisation or infection is to be inhibited, inflammation or damage associated with the bacterial infection is to be reduced, or the disease or condition associated with infection by the bacteria is to be treated. A suitable method for the production of hyperimmune colostrum is as described in Davidson et al. (1989) Lancet 2: 709-712.

For example, to inhibit the colonisation or infection by Helicobacter pylori in the gastrointestinal tract, hyperimmune colostrum from cows inoculated with Helicobacter pylori may be used.

Accordingly, in a preferred from the present invention provides a method for inhibiting colonisation of the gastrointestinal tract by Helicobacter pylori, the method including the step of administering an effective amount of a mucolytic agent and hyperimmune colostrum.

In a further preferred form, the present invention provides a method for inhibiting colonisation of the gastrointestinal tract by Helicobacter pylori, the method including the step of administering an effective amount of N-acetyl cysteine and hyperimmune colostrum.

The hyperimmune milk according to the various forms of the present invention may be any hyperimmune milk that is secreted by a mammal, including human hyperimmune milk, bovine hyperimmune milk, ovine hyperimmune milk, caprine hyperimmune milk, porcine hyperimmune milk, or equine hyperimmune milk. Preferably, the hyperimmune milk is bovine hyperimmune milk.

As will be appreciated, the hyperimmune milk is milk secreted from a mammal that has been successively immunized with the relevant bacteria (or antigens derived from the bacteria) for which colonisation or infection is to be inhibited, inflammation or damage associated with the bacterial infection is to be reduced, or the disease or condition associated with infection by the bacteria is to be treated. A suitable method for the production of hyperimmune milk is as described in Davidson et al. (1989) Lancet 2: 709-712.

For example, to inhibit the colonisation or infection by Helicobacter pylori in the gastrointestinal tract, hyperimmune milk from cows inoculated with Helicobacter pylori may be used.

Accordingly, in a preferred form the present invention provides a method for inhibiting colonisation of the gastrointestinal tract by Helicobacter pylori, the method including the step of administering an effective amount of a mucolytic agent and hyperimmune milk.

The component of colostrum and/or hyperimmune milk according to the various forms of the present invention may be one or more components derived from colostrum, hyperimmune colostrum or hyperimmune milk that is capable of acting in combination with the mucolytic agent to inhibit colonisation or infection by the relevant bacteria, reduce inflammation or damage associated with infection by the relevant bacteria, or treat a disease or condition associated with infection by the relevant bacteria. As will be appreciated, such a component includes any fraction or extract derived from colostrum, hyperimmune colostrum or hyperimmune milk by methods known in the art, any purified or semi-purified component derived from colostrum, hyperimmune colostrum or hyperimmune milk, or any component normally present in colostrum, hyperimmune colostrum or hyperimmune milk that is produced by another means, including recombinant DNA technology, or any component derived from colostrum, hyperimmune colostrum or hyperimmune milk that is further treated or modified, including hydrolysis of such components.

Preferably, the component of colostrum and/or hyperimmune milk is lactoferrin. More preferably, the component is bovine lactoferrin. In this regard, it has also been found that lactoferrin, a component of colostrum and other milk products, shows improved capacity to inhibit the colonisation or infection of mucous epithelium by H. pylori when used in combination with the mucolytic agent N-acetyl cysteine.

The component of colostrum and/or hyperimmune milk in the various forms of the present invention may also be one or more specific or cross-reactive antibodies to the bacteria, including IgG1, IgG2, IgA, IgM or antibodies.

Accordingly, in a preferred form the present invention provides a method for inhibiting bacterial colonisation of mucous epithelium in a biological system, the method including the step of administering to the biological system an effective amount of a mucolytic agent and one or more specific or cross-reactive antibodies to the bacteria colonising the mucous epithelium.

For example, in the case of inhibiting the colonisation of the gastrointestinal tract by H. pylori, one or more specific antibodies to H. pylori may be administered in combination with the mucolytic agent, or one or more antibodies that cross-react with H. pylori may be administered in combination with the mucolytic agent.

In this regard, the one or more specific or cross-reactive antibodies may be present in a mixture of other compounds, such as are present in colostrum, hyperimmune colostrum, milk, or hyperimmune milk. Alternatively, the antibodies may be in a substantially purified form, purified by a method known in the art, such as affinity purification of the antibodies from sources such as plasma, colostrum, hyperimmune colostrum, milk, hyperimmune milk, egg yolk, or hyperimmune egg yolk.

Antibodies may be raised in a human, animal or bird by a method known in the art by inoculating with the appropriate bacteria, or one or more antigens derived from the bacteria.

The amount and form of mucolytic agent to be administered in the various forms of the present invention is not particularly limited, so long as it is within such an amount, and in such a form, that generally exhibits a useful effect.

The amount of colostrum, hyperimmune milk, or component of colostrum and/or hyperimmune milk to be administered is also not particularly limited, so long as it is within such an amount, and in such a form, that generally exhibits a useful effect.

In this regard, a dose of the mucolytic agent and of one or more of colostrum, hyperimmune milk, or a component of colostrum and/or hyperimmune milk may be appropriately chosen, depending upon the particular mucolytic agent and the colostrum, hyperimmune milk or components used, the extent of bacterial colonisation or infection to be inhibited, the extent of inflammation or damage of mucous epithelium to be reduced, the tissue or organ colonised or infected, the kind of diseases or conditions to be treated, the age and body weight of the subject, the frequency of administration, or the presence of other active agents.

The mucolytic agent and one or more of colostrum, hyperimmune milk, or a component of colostrum and/or hyperimmune milk may be co-administered, or alternatively, be administered separately (so as to reach the desired site of action in combination) and either used alone or in conjunction with other agents to increase the likelihood of eradication of bacteria. Smaller doses may be applicable when used as an adjunctive therapy.

The administration of mucolytic agent and one or more of colostrum, hyperimmune milk, or a component of colostrum and/or hyperimmune milk in the various forms of the present invention may be within any time suitable to produce the desired effect. For example, the administration may be prior to the onset of colonization, so as to prevent colonization. Alternatively, the administration may be during or after colonisation of mucous epithelium has occurred or been detected.

In another preferred form, the present invention provides a method for preventing bacterial colonisation of mucous epithelium in a biological system, the method including the step of administering to the biological system an effective amount of a mucolytic agent and one or more of colostrum, hyperimmune milk, or a component of colostrum and/or hyperimmune milk that is capable of preventing bacterial colonisation in combination with the mucolytic agent.

In a human or animal system, the mucolytic agent and one or more of colostrum, hyperimmune milk, or a component of colostrum and/or hyperimmune milk may be administered orally, or by any other suitable means, and therefore transit time of the mucolytic agent and colostrum, hyperimmune milk, or a component of colostrum and/or hyperimmune milk must be taken into account. Preferably, administration of the mucolytic agent and one or more of colostrum, hyperimmune milk, or a component of colostrum and/or hyperimmune milk is by oral administration.

For oral administration of mucolytic agent and one or more of colostrum, hyperimmune milk, or a component of colostrum and/or hyperimmune milk for inhibiting the colonisation or infection of the mucous epithelium of the gastrointestinal tract, preferably the mucolytic agent is N-acetyl cysteine and the colostrum is hyperimmune colostrum. For example, to inhibit the colonisation of the gastrointestinal tract with H. pylori, the mucolytic agent is preferably N-acetyl cysteine and the colostrum is hyperimmune colostrum from cows immunised with H. pylori or antigens of H. pylori.

The administration of mucolytic agent and one or more of colostrum, hyperimmune milk, or a component of colostrum and/or hyperimmune milk in the various forms of the present invention may also include the use of one or more acceptable additives, including acceptable salts, amino acids, polypeptides, polymers, solvents, buffers, excipients and bulking agents, taking into consideration the particular physical and chemical characteristics of both the mucolytic agent and one or more of colostrum, hyperimmune milk, or a component of colostrum and/or hyperimmune milk to be administered.

For example, the mucolytic agent and one or more of colostrum, hyperimmune milk, or a component of colostrum and/or hyperimmune milk can be prepared into a variety of preparations in the form of, e.g., a food additive, an aqueous solution, an oily preparation, a fatty emulsion, an emulsion, a gel, etc., and these preparations can be administered orally, by adsorption or absorption, topically, rectally, nasally, bucally, or vaginally in dosage formulations containing conventional non-toxic acceptable carriers, or by any other convenient dosage form.

In the case of oral administration, the composition may be administered in the form of suitable oral preparations (for example solid preparations such as tablets, capsules, food additives, granules or powders; liquid preparations such as dairy products, syrup, emulsions or suspensions).

Compositions containing the mucolytic agent and one or more of colostrum, hyperimmune milk, or a component of colostrum and/or hyperimmune milk may also contain a preservative, stabiliser, dispersing agent, pH controller or isotonic agent. Examples of suitable preservatives are glycerol, propylene glycol, phenol or benzyl alcohol. Examples of suitable stabilisers are dextran, gelatin, α-tocopherol acetate or alpha-thioglycerin. Examples of suitable dispersing agents include polyoxyethylene (20), sorbitan mono-oleate (Tween 80), sorbitan sesquioleate (Span 30), polyoxyethylene (160) polyoxypropylene (30) glycol (Pluronic F68) or polyoxyethylene hydrogenated castor oil 60. Examples of suitable pH controllers include hydrochloric acid, sodium hydroxide and the like. Examples of suitable isotonic agents are glucose, D-sorbitol or D-mannitol.

The administration of the mucolytic agent and one or more of colostrum, hyperimmune milk, or a component of colostrum and/or hyperimmune milk in the various forms of the present invention may also be in the form of a composition containing an acceptable carrier, diluent, excipient, suspending agent, lubricating agent, adjuvant, vehicle, delivery system, emulsifier, disintegrant, absorbent, preservative, surfactant, colorant, flavorant or sweetener, taking into account the physical and chemical properties of the particular mucolytic agent and the colostrum, hyperimmune milk, or a component of colostrum and/or hyperimmune milk used.

When administered orally, the composition will usually be formulated into unit dosage forms such as liquids, including long life liquid formulations for oral or topical administration, aqueous suspensions or solutions, tablets, cachets, powder, granules, beads, chewable lozenges, food additives, capsules, or similar dosage forms, using conventional equipment and techniques known in the art. Such formulations typically include a liquid, solid or semisolid carrier. Exemplary carriers include lactose, dextrose, sucrose, sorbitol, mannitol, starches, gum acacia, calcium phosphate, mineral oil, cocoa butter, oil of theobroma, alginates, tragacanth, gelatin, syrup, methyl cellulose, polyoxyethylene sorbitan monolaurate, methyl hydroxybenzoate, propyl hydroxybenzoate, talc, magnesium stearate, and the like.

In the case where the composition is administered as a tablet, the tablet may be made by compressing or moulding the active ingredient, with one or more accessory ingredients optionally included. Compressed tablets may be prepared by compressing, in a suitable machine, the active ingredient in a free-flowing form such as a powder or granules, optionally mixed with a binder, lubricant, inert diluent, surface active, or dispersing agent. Moulded tablets may be made in a suitable machine, by moulding together a mixture of the powdered active ingredient and a suitable carrier, moistened with an inert liquid diluent.

The carrier may also contain minor amounts of additives, such as substances that enhance solubility, isotonicity, and chemical stability, for example anti-oxidants, buffers and preservatives.

The administration of the mucolytic agent and one or more of colostrum, hyperimmune milk, or a component of colostrum and/or hyperimmune milk in the forms of the present invention may also utilize controlled release technology. The mucolytic agent and one or more of colostrum, hyperimmune colostrum, hyperimmune milk (or one or more components of any of the foregoing) may also be administered as a sustained-release product. To further increase the sustained release effect, the composition may be formulated with additional components such as vegetable oil (for example soybean oil, sesame oil, camellia oil, castor oil, peanut oil, rape seed oil); middle fatty acid triglycerides; fatty acid esters such as ethyl oleate; polysiloxane derivatives; alternatively, water-soluble high molecular weight compounds such as hyaluronic acid or salts thereof (weight average molecular weight: ca. 80,000 to 2,000,000), carboxymethylcellulose sodium (weight average molecular weight: ca. 20,000 to 400,000), hydroxypropylcellulose (viscosity in 2% aqueous solution: 3 to 4,000 cps), atherocollagen (weight average molecular weight: ca. 300,000), polyethylene glycol (weight average molecular weight: ca. 400 to 20,000), polyethylene oxide (weight average molecular weight: ca. 100,000 to 9,000,000), hydroxypropylmethylcellulose (viscosity in 1% aqueous solution: 4 to 100,000 cSt), methylcellulose (viscosity in 2% aqueous solution: 15 to 8,000 cSt), polyvinyl alcohol (viscosity: 2 to 100 cSt), polyvinylpyrrolidone (weight average molecular weight: 25,000 to 1,200,000).

The administration of the mucolytic agent and one or more of colostrum, hyperimmune milk, or a component of colostrum and/or hyperimmune milk in the various forms of the present invention may further include the administration of other agents, including antibiotics and acid-suppressing agents.

Determination of the ability of a mucolytic agent and one or more of colostrum, hyperimmune milk, or a component of colostrum and/or hyperimmune milk to inhibit colonisation of mucous epithelium may be confirmed by a suitable method known in the art. For example, the extent of inhibition of colonisation may be determined by directly visualizing the number of bacteria in a particular sample of mucous epithelium either with or without exposure to mucolytic agent and one or more of colostrum, hyperimmune milk, or a component of colostrum and/or hyperimmune milk. In this case, the bacteria may be visualized by staining with a stain specific for detecting the bacteria of interest (for example, Giemsa staining of H. pylori), for example as described in “Saunders Dictionary and Encyclopedia of Laboratory Medicine and Technology” (Benington J. L).

Alternatively, bacterial colonisation may be determined by the extent of infiltration of the mucous sample by inflammatory cells in a particular sample of mucous epithelium with and without exposure to mucolytic agent and one or more of colostrum, hyperimmune milk, or a component of colostrum and/or hyperimmune milk, for example as described in “Histology a Text and Atlas” (Third Edition; Ross, M. H., Rommell, L. J., Kaye, G. I. 1995 Williams and Wilkins Maryland, USA).

A further method for determining the ability of a mucolytic agent and one or more of colostrum, hyperimmune milk, or a component of colostrum and/or hyperimmune milk to inhibit colonisation of mucous epithelium may be by the use of an enzyme marker such as myeloperoxidase as an index for neutrophil infiltration of the mucous epithelium, for example as described in Krawisz, J. et al. (1984) Gastroenterology 87:1344-1350. The samples of mucous epithelium for testing can be obtained by a suitable method known in the art, including biopsy of the mucous epithelium.

Preferably, the extent of inhibition of colonisation is such that the average number of bacteria per unit area of the mucous epithelium after treatment is reduced by 70% or more, as compared to the average number of bacteria per unit area in an untreated subject. More preferably, the extent of inhibition of colonisation is such that the average number of bacteria per unit area of the mucous epithelium after treatment is reduced by 85% or more, as compared to the average number of bacteria per unit area in an untreated subject. Most preferably, the extent of inhibition of colonisation is such that the average number of bacteria per unit area of the mucous epithelium after treatment is reduced by 90% or more, as compared to the average number of bacteria per unit area in an untreated subject.

It has also been found that lactoferrin, an anti-bacterial component of colostrum and milk, shows improved capacity to inhibit the colonisation or infection of mucous epithelium by bacteria. For example, lactoferrin shows improved capacity to inhibit colonisation of mucous epithelium by H. pylori when used in combination with the mucolytic agent N-acetyl cysteine.

Accordingly, in a preferred form the present invention provides a method for inhibiting bacterial colonisation of mucous epithelium in a biological system, the method including the step of administering to the biological system an effective amount of a mucolytic agent and an anti-bacterial agent derived from a milk product.

The anti-bacterial agent derived from a milk product is any component of milk, hyperimmune milk, colostrum, hyperimmune colostrum or any other milk derived product that has anti-bacterial activity (either bactericidal or bacteriostatic) produced by a method known in the art. This includes one or more fractions or extracts derived from milk, hyperimmune milk, colostrum or hyperimmune colostrum, or any component with anti-bacterial activity in a composition that would normally be present in milk, hyperimmune milk, colostrum or hyperimmune colostrum, including substantially purified products from milk, hyperimmune milk, colostrum or hyperimmune colostrum, or a product produced by recombinant DNA technology.

A suitable method for determining the anti-bacterial properties of an agent derived from milk is as described in Korhonen et al. (1995) Journal of Applied Bacteriology 78: 655-662.

For example, the anti-bacterial agent derived from a milk product in the various forms of the present invention may be lactoferrin, lactoperoxidase, lysozyme or immunoglobulins, including IgG1, IgG2, IgA, IgM, or an antibacterial peptide or anti-bacterial sugar present in a milk product.

As will be appreciated, the anti-bacterial agent in the various forms of the present invention could also be derived from a product not derived from milk. For example, the anti-bacterial agent could be an IgY antibody (the equivalent of IgG1) derived from hyperimmune egg yolks.

Preferably, the anti-bacterial agent derived from a milk product is lactoferrin. The lactoferrin may be bovine lactoferrin, ovine lactoferrin, caprine colostrum, porcine lactoferrin, equine lactoferrin or human lactoferrin. Most preferably, the lactoferrin is bovine lactoferrin. The lactoferrin may be isolated from colostrum or milk in a semi-purified or substantially pure form by a suitable method known in the art. Alternatively, the lactoferrin may be recombinant lactoferrin, produced by expressing the gene for lactoferrin in an appropriate expression system.

Accordingly, in a further preferred form, the present invention provides a method for inhibiting bacterial colonisation of mucous epithelium in a biological system, the method including the step of administering to the biological system an effective amount of a mucolytic agent and lactoferrin.

The lactoferrin in the various forms of the present invention may be further treated to improve its activity to inhibit colonisation or infection of mucous epithelium by bacteria. For example, the lactoferrin may be proteolytically digested to produce a protein fragment or polypeptide that displays improved activity with respect to inhibiting colonisation. To produce such a fragment, a protease such as pepsin may be used to digest the lactoferrin under acidic conditions. Alternatively, the lactoferrin may be treated by thermal inactivation at acidic pH, or a specific fragment of lactoferrin may be produced by recombinant means.

In this regard, it has also been found that hydrolysed bovine lactoferrin, which is produced upon the digestion of bovine lactoferrin with gastric pepsin or by thermal inactivation at acidic pH, shows improved activity to inhibit colonisation or infection of mucous epithelium by H. pylori when used in combination with a mucolytic agent such as N-acetyl cysteine.

Accordingly, in another preferred form, the present invention provides a method for inhibiting bacterial colonisation of mucous epithelium in a biological system, the method including the step of administering to the biological system an effective amount of a mucolytic agent and hydrolysed lactoferrin.

As will be appreciated, the same considerations relevant to the administration of mucolytic agent and one or more of colostrum, hyperimmune milk or a component of colostrum and/or hyperimmune milk as discussed previously also apply to the administration of mucolytic agent and an anti-bacterial agent derived from colostrum/milk.

The method of this form of the present invention may also include the administration of other agents, including antibiotics and acid-suppressing agents.

Preferably, the method of this form of the present invention also includes the administration of an antibiotic. An example of a suitable antibiotic is amoxicillin.

Accordingly, in another form the present invention provides a method for inhibiting bacterial colonisation of mucous epithelium in a biological system, the method including the step of administering to the biological system an effective amount of a mucolytic agent, an antibiotic and one or more of colostrum, hyperimmune milk, or a component of colostrum and/or hyperimmune milk that is capable of inhibiting bacterial colonisation in combination with the mucolytic agent.

In this regard, the method of this form of the present invention also reduces the likelihood of antibiotic resistance developing when antibiotic therapy is used.

Accordingly, the present invention also provides a method of reducing the development of antibiotic resistant bacteria in a biological system, the method including the step of administering to the biological system an effective amount of a mucolytic agent, and one or more of colostrum, hyperimmune milk, or a component of colostrum and/or hyperimmune milk.

The present invention also provides a method for reducing bacterial infection of mucous epithelium in a biological system, the method including the step of administering to the biological system an effective amount of a mucolytic agent and one or more of colostrum, hyperimmune milk, or a component of colostrum and/or hyperimmune milk that is capable of reducing bacterial infection in combination with the mucolytic agent.

Determination of the ability of a mucolytic agent and one or more of colostrum, hyperimmune milk, or a component of colostrum and/or hyperimmune milk to reduce infection of mucous epithelium may be confirmed by a suitable method known in the art. For example, the extent of reduction of infection may be determined by directly visualizing the number of bacteria in a particular sample of mucous epithelium either with or without exposure to mucolytic agent and one or more of colostrum, hyperimmune milk, or a component of colostrum and/or hyperimmune milk. In this case, the bacteria may be visualized by staining with a stain specific for detecting the bacteria of interest (for example, Giemsa staining of H. pylori) as described in “Saunders Dictionary and Encyclopedia of Laboratory Medicine and Technology” (Benington J. L).

Alternatively, bacterial infection may be determined by the extent of infiltration of the mucous sample by inflammatory cells in a particular sample of mucous epithelium with and without exposure to mucolytic agent and one or more of colostrum, hyperimmune milk, or a component of colostrum and/or hyperimmune milk, for example as described in “Histology a Text and Atlas” (Third Edition; Ross, M. H., Rommell, L. J., Kaye, G. I. 1995 Williams and Wilkins Maryland, USA).

A further method for determining the ability of a mucolytic agent and one or more of colostrum, hyperimmune milk, or a component of colostrum and/or hyperimmune milk to reduce infection of mucous epithelium may be by the use of an enzyme marker such as myeloperoxidase as an index for neutrophil infiltration of the mucous epithelium, for example as described in Krawisz, J. et al (1984) Gastroenterology 87:1344-1350. The samples of mucous epithelium for testing can be obtained by a suitable method known in the art, including biopsy of the mucous epithelium.

Preferably, the extent of reduction of infection is such that the average number of bacteria per unit area of the mucous epithelium after treatment is reduced by 50% or more, as compared to the average number of bacteria per unit area in an untreated subject. More preferably, the extent of reduction of infection is such that the average number of bacteria per unit area of the mucous epithelium after treatment is reduced by 60% or more, as compared to the average number of bacteria per unit area in an untreated subject. Most preferably, the extent of reduction of infection is such that the average number of bacteria per unit area of the mucous epithelium after treatment is reduced by 80% or more, as compared to the average number of bacteria per unit area in an untreated subject.

In a particularly preferred form, the present invention also provides a method for reducing bacterial infection of mucous epithelium in a biological system, the method including the step of administering to the biological system an effective amount of N-acetyl cysteine and hyperimmune colostrum.

In another preferred form, the present invention also provides a method for reducing bacterial infection of mucous epithelium in a biological system, the method including the step of administering to the biological system an effective amount of a mucolytic agent and an anti-bacterial agent derived from milk.

In a further preferred form, the present invention provides a method for reducing bacterial infection of mucous epithelium in a biological system, the method including the step of administering to the biological system an effective amount of a mucolytic agent and lactoferrin.

In another preferred form, the present invention provides a method for reducing bacterial infection of mucous epithelium in a biological system, the method including the step of administering to the biological system an effective amount of a mucolytic agent and hydrolysed lactoferrin.

In another preferred form the present invention provides a method for reducing bacterial infection of mucous epithelium in a biological system, the method including the step of administering to the biological system an effective amount of a mucolytic agent and one or more specific or cross-reactive antibodies to the bacteria infecting the mucous epithelium.

As discussed previously, the administration of mucolytic agent and one or more of colostrum, hyperimmune milk, or a component of colostrum and/or hyperimmune milk in the various forms of the present invention may be within any time suitable to produce the desired effect. For example, the administration may be prior to the onset of infection of the mucous epithelium. Alternatively, the administration may be during or after infection of the mucous epithelium has occurred or been detected.

In another preferred form, the present invention provides a method for preventing bacterial infection of mucous epithelium in a biological system, the method including the step of administering to the biological system an effective amount of a mucolytic agent and one or more of colostrum, hyperimmune milk, or a component of colostrum and/or hyperimmune milk that is capable of preventing bacterial infection in combination with the mucolytic agent.

The method of this form of the present invention may also include the administration of other agents, including antibiotics and acid-suppressing agents.

Preferably, the method of this form of the present invention also includes the administration of an antibiotic. An example of a suitable antibiotic is amoxicillin.

Accordingly, in another form the present invention provides a method for reducing bacterial infection of mucous epithelium in a biological system, the method including the step of administering to the biological system an effective amount of a mucolytic agent, an antibiotic and one or more of colostrum, hyperimmune milk, or a component of colostrum and/or hyperimmune milk that is capable of inhibiting bacterial infection in combination with the mucolytic agent.

The present invention also provides a method for reducing inflammation of mucous epithelium associated with bacterial infection in a biological system, the method including the step of administering to the biological system an effective amount of a mucolytic agent and one or more of colostrum, hyperimmune milk, or a component of colostrum and/or hyperimmune milk that is capable of reducing inflammation of mucous epithelium associated with bacterial infection in combination with the mucolytic agent.

Determination of the ability of a mucolytic agent and one or more of colostrum, hyperimmune milk, or a component of colostrum and/or hyperimmune milk to reduce inflammation of mucous epithelium associated with bacterial infection may be confirmed by a suitable method known in the art. For example, the extent of inflammation may be determined by the extent of infiltration of the mucous sample by inflammatory cells in a particular sample of mucous epithelium with and without exposure to mucolytic agent and one or more of colostrum, hyperimmune milk or a component of colostrum and/or hyperimmune milk as described in “Histology a Text and Atlas” (Third Edition; Ross, M. H., Rommell, L. J., Kaye, G. I. 1995 Williams and Wilkins Maryland, USA).

Alternatively, the extent of inflammation may be determined by use of an enzyme marker such as myeloperoxidase as an index for neutrophil infiltration of the mucous epithelium, for example as described in Krawisz, J. et al. (1984) Gastroenterology 87:1344-1350. The samples of mucous epithelium for testing can be obtained by a suitable method known in the art, including biopsy of the mucous epithelium.

Preferably, the extent of reduction of inflammation is such that the level of myeloperoxidase activity in a tissue sample of the mucous epithelium after treatment is reduced by 50% or more, as compared to the myeloperoxidase activity in a tissue sample from an untreated subject. More preferably, the extent of reduction of inflammation is such that the level of myeloperoxidase activity in a tissue sample of the mucous epithelium after treatment is reduced by 65% or more, as compared to the myeloperoxidase activity in a tissue sample from an untreated subject.

The inflammation of mucous epithelium associated with bacterial infection in the various forms of the present invention may be any inflammation of mucous epithelium that is part of one or more of the following organs or tissues: stomach, including the cardia, fundus, body, antrum and pylorus of the stomach; duodenum; ileum; small intestine; large intestine; colon; bowel; rectum; esophagus; mouth; tongue; pharynx; urino-genital tact; eye; and respiratory tract, including the nasal cavity, oral cavity, larynx, trachea, bronchi including bronchioles and alveoli, and lungs. Preferably, the inflammation of mucous epithelium associated with bacterial infection is inflammation of mucous epithelium of an animal or human. Most preferably, the inflammation of mucous epithelium is inflammation of mucous epithelium of a human.

Preferably, the inflammation of mucous epithelium associated with bacterial infection is inflammation of mucous epithelium associated with the following diseases or conditions: gastric inflammation; ulcers of the stomach or duodenum; non-ulcer dyspepsia; gastric conditions associated with leukocyte infiltration; urinary tract infections; strep throat; infective endocarditis; bacterial pneumonia; whooping cough; gingivitis; acute or chronic bronchitis; bronchiectasis; asthmatic bronchitis; bronchial asthma; bronchiolitis; cystic fibrosis; laryngopharyngitis; acute or chronic rhinitis. Preferably, the inflammation of mucous epithelium associated with bacterial infection is inflammation of mucous epithelium associated with gastric inflammation, ulcers of the stomach or duodenum, non-ulcer dyspepsia, or a gastric condition associated with leukocyte infiltration.

In a preferred form, the present invention also provides a method for reducing inflammation of mucous epithelium associated with bacterial infection in a biological system, the method including the step of administering to the biological system an effective amount of a mucolytic agent and an anti-bacterial agent derived from a milk product.

In a further preferred form, the present invention provides a method for reducing inflammation of mucous epithelium associated with bacterial infection in a biological system, the method including the step of administering to the biological system an effective amount of a mucolytic agent and lactoferrin.

In another preferred form, the present invention provides a method for reducing inflammation of mucous epithelium associated with bacterial infection in a biological system, the method including the step of administering to the biological system an effective amount of a mucolytic agent and hydrolysed lactoferrin.

In another preferred form the present invention provides a method for reducing inflammation of mucous epithelium associated with bacterial infection in a biological system, the method including the step of administering to the biological system an effective amount of a mucolytic agent and one or more specific or cross-reactive antibodies to the bacteria infecting the mucous epithelium.

As will be appreciated, the same considerations relevant to the administration of mucolytic agent and one or more of colostrum, hyperimmune milk, or a component of colostrum and/or hyperimmune milk in regard to inhibiting bacterial colonisation or infection of mucous epithelium discussed above will also apply to the administration of mucolytic agent and an anti-bacterial agent derived from a milk product, the administration of a mucolytic agent and lactoferrin, or the administration of a mucolytic agent and hydrolysed lactoferrin in regard to reducing inflammation of mucous epithelium associated with bacterial infection.

As discussed previously, the administration of mucolytic agent and one or more of colostrum, hyperimmune milk, or a component of colostrum and/or hyperimmune milk in the various forms of the present invention may be within any time suitable to produce the desired effect. For example, the administration may be prior to the onset of inflammation of mucous epithelium associated with bacterial infection of the mucous epithelium. Alternatively, the administration may be during or after the onset of inflammation of mucous epithelium associated with bacterial infection of the mucous epithelium has occurred or been detected.

In another preferred form, the present invention provides a method for preventing inflammation of mucous epithelium associated with bacterial infection in a biological system, the method including the step of administering to the biological system an effective amount of a mucolytic agent and one or more of colostrum, hyperimmune milk, or a component of colostrum and/or hyperimmune milk that is capable of preventing inflammation of mucous epithelium associated with bacterial infection in combination with the mucolytic agent.

The method of this form of the present invention may also include the administration of other agents, including antibiotics and acid-suppressing agents.

Preferably, the method of this form of the present invention also includes the administration of an antibiotic. An example of a suitable antibiotic is amoxicillin.

Accordingly, in another form the present invention provides a method for reducing inflammation of mucous epithelium associated with bacterial infection in a biological system, the method including the step of administering to the biological system an effective amount of a mucolytic agent, an antibiotic and one or more of colostrum, hyperimmune milk, or a component of colostrum and/or hyperimmune milk that is capable of reducing inflammation of mucous epithelium associated with bacterial infection in combination with the mucolytic agent.

In this regard, the method of this form of the present invention also reduces the likelihood of antibiotic resistance developing when antibiotic therapy is used.

The present invention also provides a method for reducing damage to mucous epithelium associated with bacterial infection of the mucous epithelium in a biological system, the method including the step of administering to the biological system an effective amount of a mucolytic agent and one or more of colostrum, hyperimmune milk, or a component of colostrum and/or hyperimmune milk that is capable of reducing the damage to mucous epithelium associated with bacterial infection in combination with the mucolytic agent.

Determination of the ability of a mucolytic agent and one or more of colostrum, hyperimmune milk, or a component of colostrum and/or hyperimmune milk to reduce damage to mucous epithelium associated with bacterial infection may be confirmed by a suitable method known in the art, for example as described in “Histology a Text and Atlas” (Third Edition; Ross, M. H., Rommell, L. J., Kaye, G. I. 1995 Williams and Wilkins Maryland, USA). For example, the extent of damage may be determined by histopathological examination of tissue samples with and without exposure to mucolytic agent and one or more of colostrum, hyperimmune milk or a component of colostrum and/or hyperimmune milk.

The damage to mucous epithelium associated with bacterial infection in the various forms of the present invention may be any damage to mucous epithelium that is part of one or more of the following organs or tissues: the gastrointestinal tract; stomach, including the cardia, fundus, body, antrum and pylorus of the stomach; duodenum; ileum; small intestine; large intestine; colon; bowel; rectum; esophagus; mouth; tongue; pharynx; urino-genital tact; eye; and respiratory tract, including the nasal cavity, larynx, trachea, bronchi including bronchioles and alveoli, and lungs. Preferably, the damage to mucous epithelium associated with bacterial infection is damage to mucous epithelium of an animal or human. Most preferably, the damage to mucous epithelium is inflammation of mucous epithelium of a human.

Preferably, the damage to mucous epithelium associated with bacterial infection is damage to mucous epithelium associated with the following diseases or conditions: gastric inflammation; ulcers of the stomach or duodenum; non-ulcer dyspepsia; gastric conditions associated with leukocyte infiltration; urinary tract infections; strep throat; infective endocarditis; bacterial pneumonia; whooping cough; gingivitis; acute or chronic bronchitis; bronchiectasis; asthmatic bronchitis; bronchial asthma; bronchiolitis; cystic fibrosis; laryngopharyngitis; acute or chronic rhinitis. Preferably, the damage to mucous epithelium associated with bacterial infection is damage of mucous epithelium associated with gastric inflammation, ulcers of the stomach or duodenum, non-ulcer dyspepsia, or a gastric condition associated with leukocyte infiltration.

In a particularly preferred form, the present invention provides a method for reducing damage to mucous epithelium associated with bacterial infection of the mucous epithelium in a biological system, the method including the step of administering to the biological system an effective amount of N-acetyl cysteine and hyperimmune colostrum.

In a preferred form, the present invention also provides a method for reducing damage to mucous epithelium associated with bacterial infection in a biological system, the method including the step of administering to the biological system an effective amount of a mucolytic agent and an anti-bacterial agent derived from a milk product.

In a further preferred form, the present invention provides a method of reducing damage to mucous epithelium associated with bacterial infection in a biological system, the method including the step of administering to the biological system an effective amount of a mucolytic agent and lactoferrin.

In a further preferred form, the present invention provides a method of reducing damage to mucous epithelium associated with bacterial infection in a biological system, the method including the step of administering to the biological system an effective amount of a mucolytic agent and hydrolysed lactoferrin.

In another preferred form the present invention provides a method for reducing damage to mucous epithelium associated with bacterial infection in a biological system, the method including the step of administering to the biological system an effective amount of a mucolytic agent and one or more specific or cross-reactive antibodies to the bacteria infecting the mucous epithelium.

As will be appreciated, the same considerations relevant to the administration of mucolytic agent and one or more of colostrum, hyperimmune milk, or a component of colostrum and/or hyperimmune milk in regard to inhibiting bacterial colonisation or infection of mucous epithelium discussed above will also apply to the administration of mucolytic agent and an anti-bacterial agent derived from a milk product, the administration of a mucolytic agent and lactoferrin, or the administration of a mucolytic agent and hydrolysed lactoferrin in regard to reducing damage to mucous epithelium associated with bacterial infection.

As discussed previously, the administration of mucolytic agent and one or more of colostrum, hyperimmune milk, or a component of colostrum and/or hyperimmune milk in the various forms of the present invention may be within any time suitable to produce the desired effect. For example, the administration may be prior to the onset of damage to mucous epithelium associated with bacterial infection of the mucous epithelium. Alternatively, the administration may be during or after the onset of damage to mucous epithelium associated with bacterial infection of the mucous epithelium has occurred or been detected.

In another preferred form, the present invention provides a method for preventing damage to mucous epithelium associated with bacterial infection in a biological system, the method including the step of administering to the biological system an effective amount of a mucolytic agent and one or more of colostrum, hyperimmune milk, or a component of colostrum and/or hyperimmune milk that is capable of preventing the damage to mucous epithelium associated with bacterial infection in combination with the mucolytic agent.

The method of this form of the present invention may also include the administration of other agents, including antibiotics and acid-suppressing agents.

Preferably, the method of this form of the present invention also includes the administration of an antibiotic. An example of a suitable antibiotic is amoxicillin.

Accordingly, in another form the present invention provides a method for reducing damage to mucous epithelium associated with bacterial infection of the mucous epithelium in a biological system, the method including the step of administering to the biological system an effective amount of a mucolytic agent, an antibiotic and one or more of colostrum, hyperimmune milk, or a component of colostrum and/or hyperimmune milk that is capable of reducing damage to mucous epithelium associated with bacterial in combination with the mucolytic agent.

In this regard, the method of this form of the present invention also reduces the likelihood of antibiotic resistance developing when antibiotic therapy is used.

The present invention also provides a method for treating a disease or condition associated with bacterial infection of mucous epithelium in a subject, the method including the step of administering to the subject an effective amount of a mucolytic agent and one or more of colostrum, hyperimmune milk, or a component of colostrum and/or hyperimmune milk that is capable of treating the disease or condition associated with bacterial infection of mucous epithelium in combination with the mucolytic agent.

The disease or condition associated with bacterial infection of mucous epithelium in the various forms of the present invention may be a disease or condition associated with the bacterial infection of the mucous epithelium of one or more of the following organs or tissues: gastrointestinal tract; stomach, including the cardia, fundus, body, antrum and pylorus of the stomach; duodenum; ileum; small intestine; large intestine; colon; bowel; rectum; esophagus; mouth; tongue; pharynx; urino-genital tact; eye; and respiratory tract, including the nasal cavity, larynx, trachea, bronchi including bronchioles and alveoli, and lungs. Preferably, the disease or condition associated with bacterial infection of mucous epithelium is a disease or condition associated with the bacterial infection of mucous epithelium of an animal or human. Most preferably, the disease or condition associated with bacterial infection of mucous epithelium is a disease or condition associated with the bacterial infection of mucous epithelium of a human.

Preferably, the disease or condition associated with bacterial infection of mucous epithelium is one of following diseases or conditions: gastric inflammation; ulcers of the stomach or duodenum; non-ulcer dyspepsia; gastric conditions associated with leukocyte infiltration; urinary tract infections; strep throat; infective endocarditis; bacterial pneumonia; whooping cough; gingivitis; acute or chronic bronchitis; bronchiectasis; asthmatic bronchitis; bronchial asthma; bronchiolitis; cystic fibrosis; laryngopharyngitis; acute or chronic rhinitis. Most preferably, the disease or condition associated with bacterial infection of mucous epithelium is gastric inflammation, ulcers of the stomach or duodenum, non-ulcer dyspepsia, and gastric conditions associated with leukocyte infiltration.

The subject in the various forms of the present invention may be any animal or human subject that is susceptible to a disease or condition associated with bacterial infection of mucous epithelium, or has a disease or condition associated with bacterial infection of mucous epithelium. Most preferably, the biological system is a human subject suffering from a disease or condition associated with bacterial infection of mucous epithelium.

In a particularly preferred form, the present invention provides a method for treating a disease or condition associated with bacterial infection of mucous epithelium in a subject, the method including the step of administering to the subject an effective amount of N-acetyl cysteine and hyperimmune colostrum.

In another preferred form, the present invention also provides a method for treating a disease or condition associated with bacterial infection of mucous epithelium in a subject, the method including the step of administering to the subject an effective amount of a mucolytic agent and an anti-bacterial agent derived from milk.

In another preferred form, the present invention provides a method for treating a disease or condition associated with bacterial infection of mucous epithelium in a subject, the method including the step of administering to the subject an effective amount of a mucolytic agent and lactoferrin.

In another preferred form, the present invention provides a method for treating a disease or condition associated with bacterial infection of mucous epithelium in a subject, the method including the step of administering to the subject an effective amount of a mucolytic agent and hydrolysed lactoferrin.

In another preferred form the present invention provides a method for treating a disease or condition associated with bacterial infection of mucous epithelium in a subject, the method including the step of administering to the subject an effective amount of a mucolytic agent and one or more specific or cross-reactive antibodies to the bacteria infecting the mucous epithelium.

As will be appreciated, the same considerations relevant to the administration of mucolytic agent and one or more of colostrum, hyperimmune milk, or a component of colostrum and/or hyperimmune milk in regard to inhibiting bacterial colonisation or infection of mucous epithelium discussed above will also apply to the administration for treating a disease or condition associated with bacterial infection of mucous epithelium.

As discussed previously, the administration of mucolytic agent and one or more of colostrum, hyperimmune milk, or a component of colostrum and/or hyperimmune milk in the various forms of the present invention may be within any time suitable to produce the desired effect. For example, the administration may be prior to the onset of a disease or condition in a subject associated with bacterial infection of the mucous epithelium. Alternatively, the administration may be during or after the onset of a disease or condition associated with bacterial infection of the mucous epithelium has occurred or been detected.

In another preferred form, the present invention provides a method for preventing a disease or condition associated with bacterial infection of mucous epithelium in a subject, the method including the step of administering to the subject an effective amount of a mucolytic agent and one or more of colostrum, hyperimmune milk, or a component of colostrum and/or hyperimmune milk that is capable of preventing the disease or condition associated with bacterial infection of mucous epithelium in combination with the mucolytic agent.

In another form, the present invention provides the use of an effective amount of a mucolytic agent and one or more of colostrum, hyperimmune milk, or a component of colostrum and/or hyperimmune milk for the preparation of a medicament for the treatment of a disease or condition associated with bacterial infection of mucous epithelium.

The method of this form of the present invention may also include the administration of other agents, including antibiotics and acid-suppressing agents.

Preferably, the method of this form of the present invention also includes the administration of an antibiotic. An example of a suitable antibiotic is amoxicillin.

Accordingly, in another form the present invention provides a method for preventing and/or treating a disease or condition associated with bacterial infection of mucous epithelium in a subject, the method including the step of administering to the biological system an effective amount of a mucolytic agent, an antibiotic and one or more of colostrum, hyperimmune milk, or a component of colostrum and/or hyperimmune milk that is capable of preventing and/or treating a disease associated with bacterial infection of mucous epithelium in combination with the mucolytic agent.

In this regard, the method of this form of the present invention also reduces the likelihood of antibiotic resistance developing when antibiotic therapy is used.

The present invention also provides a composition including a mucolytic agent and one or more of colostrum, hyperimmune milk, or a component of colostrum and/or hyperimmune milk.

The amount of the mucolytic agent to be used in the composition is not particularly limited, so long as it is within such an amount that generally will exhibit a therapeutic effect when the composition is administered to a subject.

The amount of one or more of colostrum, hyperimmune milk, or a component of colostrum and/or hyperimmune milk to be used in the composition is also not particularly limited, so long as it is within such an amount that generally will exhibit an useful effect when the composition is administered to a subject.

In this regard, a dose of the mucolytic agent and one or more of colostrum, hyperimmune milk, or a component of colostrum and/or hyperimmune milk used in the composition may be appropriately chosen, depending upon the particular mucolytic agent and colostrum, hyperimmune milk, or component of colostrum and/or hyperimmune milk used, the extent of bacterial colonisation or infection to be inhibited, the extent of inflammation of mucous epithelium to be reduced, the tissue or organ colonised or infected, the kind of diseases or conditions to be treated, the age and body weight of the subject, the frequency of administration, and the presence of other active agents.

The mucolytic agent and one or more of colostrum, hyperimmune milk, or a component of colostrum and/or hyperimmune milk may be co-administered in a single composition, or alternatively, be administered as separate compositions and thereby act at the desired site of action in combination.

In another preferred form, the present invention provides a composition for inhibiting the colonisation and/or infection of mucous epithelium by bacteria, the composition including a mucolytic agent and one or more of colostrum, hyperimmune milk, or a component of colostrum and/or hyperimmune milk that is capable of inhibiting the colonisation and/or infection of mucous epithelium by bacteria.

In another preferred form, the present invention provides a composition for treating a disease or condition associated with bacterial infection of mucous epithelium, the composition including a mucolytic agent and one or more of colostrum, hyperimmune milk, or a component of colostrum and/or hyperimmune milk that is capable of treating the disease associated with bacterial infection of mucous epithelium.

For a composition suitable for the administration of mucolytic agent and one or more of colostrum, hyperimmune milk, or a component of colostrum and/or hyperimmune milk for inhibiting and/or preventing the colonisation or infection of the mucous epithelium of the gastrointestinal tract (or treating a disease or condition associated with bacterial infection of the gastrointestinal tract), preferably the mucolytic agent in the composition is N-acetyl cysteine and the colostrum is hyperimmune colostrum. For example, to inhibit the colonisation of the gastrointestinal tract with Helicobacter pylori, the composition preferably includes N-acetyl cysteine and hyperimmune colostrum from cows immunised with H. pylori.

The composition may also include one or more acceptable additives, including salts, amino acids, polypeptides, polymers, solvents, buffers, excipients and bulking agents, taking into consideration the particular physical and chemical characteristics of the mucolytic agent and one or more of colostrum, hyperimmune milk, or a component of colostrum and/or hyperimmune milk to be administered.

For example, the mucolytic agent and one or more of colostrum, hyperimmune milk, or a component of colostrum and/or hyperimmune milk can be prepared into a variety of preparations in the form of, e.g., an aqueous solution, an oily preparation, a fatty emulsion, an emulsion, a gel, etc., and these preparations can be administered orally, by adsorption, absorption, topically, rectally, nasally, bucally, vaginally, or by any other convenient dosage form.

In the case of oral administration, the composition may be in the form of suitable oral preparations, including liquid preparations such as dairy products, syrup, emulsions or suspensions, or solid preparations such as tablets, capsules, granules or powders.

Compositions containing the mucolytic agent and one or more of colostrum, hyperimmune milk, or a component of colostrum and/or hyperimmune milk may also contain a preservative, stabiliser, dispersing agent, pH controller or isotonic agent. Examples of suitable preservatives are glycerin, propylene glycol, phenol or benzyl alcohol. Examples of suitable stabilisers are dextran, gelatin, α-tocopherol acetate or alpha-thioglycerin. Examples of suitable dispersing agents include polyoxyethylene (20), sorbitan mono-oleate (Tween 80), sorbitan sesquioleate (Span 30), polyoxyethylene (160) polyoxypropylene (30) glycol (Pluronic F68) or polyoxyethylene hydrogenated castor oil 60. Examples of suitable pH controllers include hydrochloric acid, sodium hydroxide and the like. Examples of suitable isotonic agents are glucose, D-sorbitol or D-mannitol.

The composition may further include an acceptable carrier, diluent, excipient, suspending agent, lubricating agent, adjuvant, vehicle, delivery system, emulsifier, disintegrant, absorbent, preservative, surfactant, colorant, flavorant or sweetener, taking into account the physical and chemical properties of the particular mucolytic agent and the form of the colostrum used.

When administered orally, the composition will usually be in a unit dosage form such as a liquid, including long life liquid formulations for oral or topical administration, aqueous suspensions or solutions, tablets, cachets, powder, granules, beads, chewable lozenges, food additives, capsules, or similar dosage forms, using conventional equipment and techniques known in the art. Such formulations typically include a solid, semisolid, or liquid carrier. Exemplary carriers include lactose, dextrose, sucrose, sorbitol, mannitol, starches, gum acacia, calcium phosphate, mineral oil, cocoa butter, oil of theobroma, alginates, tragacanth, gelatin, syrup, methyl cellulose, polyoxyethylene sorbitan monolaurate, methyl hydroxybenzoate, propyl hydroxybenzoate, talc, magnesium stearate, and the like.

In the case of administration involving a table, the tablet may be made by compressing or moulding the active ingredient, with one or more accessory ingredients as an option. Compressed tablets may be prepared by compressing, in a suitable machine, the active ingredient in a free-flowing form such as a powder or granules, optionally mixed with a binder, lubricant, inert diluent, surface active, or dispersing agent. Moulded tablets may be made by moulding in a suitable machine, a mixture of the powdered active ingredient and a suitable carrier moistened with an inert liquid diluent.

The carrier may contain minor amounts of additives, such as substances that enhance solubility, isotonicity, and chemical stability, for example anti-oxidants, buffers and preservatives.

The composition may also include agents to allow controlled or sustained release of the mucolytic agent and one or more of colostrum, hyperimmune milk, or a component of colostrum and/or hyperimmune milk. For example, in relation to a sustained release of the agents, the composition may be formulated with additional components such as vegetable oil (for example soybean oil, sesame oil, camellia oil, castor oil, peanut oil, rape seed oil); middle fatty acid triglycerides; fatty acid esters such as ethyl oleate; polysiloxane derivatives; alternatively, water-soluble high molecular weight compounds such as hyaluronic acid or salts thereof (weight average molecular weight: ca. 80,000 to 2,000,000), carboxymethylcellulose sodium (weight average molecular weight: ca. 20,000 to 400,000), hydroxypropylcellulose (viscosity in 2% aqueous solution: 3 to 4,000 cps), atherocollagen (weight average molecular weight: ca. 300,000), polyethylene glycol (weight average molecular weight: ca. 400 to 20,000), polyethylene oxide (weight average molecular weight: ca. 100,000 to 9,000,000), hydroxypropylmethylcellulose (viscosity in 1% aqueous solution: 4 to 100,000 cSt), methylcellulose (viscosity in 2% aqueous solution: 15 to 8,000 cSt), polyvinyl alcohol (viscosity: 2 to 100 cSt), polyvinylpyrrolidone (weight average molecular weight: 25,000 to 1,200,000).

Alternatively, the composition may have the mucolytic agent and one or more of colostrum, hyperimmune milk, or a component of colostrum and/or hyperimmune milk incorporated into a hydrophobic polymer matrix for controlled release over a period of days. The composition may then be moulded into a solid form, or externally applied patch, suitable for providing efficacious concentrations of the mucolytic agent and colostrum over a prolonged period of time without the need for frequent re-dosing. Such controlled release films are well known to the art. Other examples of polymers commonly employed for this purpose that may be used include nondegradable ethylene-vinyl acetate copolymer a degradable lactic acid-glycolic acid copolymers which may be used externally or internally. Certain hydrogels such as poly(hydroxyethylmethacrylate) or poly(vinylalcohol) also may be useful, but for shorter release cycles than the other polymer release systems, such as those mentioned above.

The carrier may also be a solid biodegradable polymer or mixture of biodegradable polymers with appropriate time-release characteristics and release kinetics. The composition may then be moulded into a solid implant suitable for providing efficacious concentrations of the mucolytic agent and one or more of colostrum, hyperimmune milk, or a component of colostrum and/or hyperimmune milk over a prolonged period of time without the need for frequent re-dosing. The mucolytic agent and/or one or more of colostrum, hyperimmune milk, or a component of colostrum and/or hyperimmune milk can be incorporated into the biodegradable polymer or polymer mixture in any suitable manner known to one of ordinary skill in the art and may form a homogeneous matrix with the biodegradable polymer, or may be encapsulated in some way within the polymer, or may be moulded into a solid implant.

The composition according to the present invention may further include other agents, including antibiotics and acid-suppressing agents.

Preferably, the composition also includes the administration of an antibiotic. An example of a suitable antibiotic is amoxicillin.

In a particularly preferred form, the present invention provides a composition including N-acetyl cysteine and hyperimmune colostrum.

In another preferred form, the present invention also provides a composition including a mucolytic agent and an anti-bacterial agent derived from a milk product.

In a further preferred form, the present invention provides a composition for inhibiting the colonisation and/or infection of mucous epithelium by bacteria, the composition including a mucolytic agent and an antibacterial agent derived from a milk product.

In another preferred form, the present invention provides a composition for treating a disease or condition associated with bacterial infection of mucous epithelium, the composition including a mucolytic agent and an antibacterial agent derived from a milk product.

The anti-bacterial agent derived from a milk product in the composition may be any component or extract isolated from milk, colostrum, hyperimmune colostrum, hyperimmune milk, or any component normally present in milk, colostrum, hyperimmune colostrum, or hyperimmune colostrum that has anti-bacterial activity. This includes one or more fractions or extracts derived from milk, colostrum, hyperimmune colostrum, or hyperimmune colostrum or any component with anti-bacterial activity that would normally be present in milk, colostrum, hyperimmune colostrum, or hyperimmune colostrum produced by methods known in the art (for example, a substantially pure component or a protein normally present that is produced by recombinant DNA methodology.

Preferably, the * anti-bacterial agent derived from a milk product in the composition is lactoferrin, lactoperoxidase, lysozyme or immunoglobulins, including IgG1, IgG2, IgA, IgM or an anti-bacterial peptide or anti-bacterial sugar present in a milk product. More preferably, the anti-bacterial agent derived from a milk product in the composition is lactoferrin. More preferably the lactoferrin in the composition is bovine lactoferrin, ovine lactoferrin, porcine lactoferrin, equine lactoferrin or human lactoferrin. Most preferably, the lactoferrin in the composition is bovine lactoferrin. The lactoferrin in the composition may be isolated from a milk product in a substantially pure form, or alternatively, be recombinant lactoferrin produced by expressing the gene for lactoferrin in an appropriate expression system.

Accordingly, in a further preferred form, the present invention also provides a composition including a mucolytic agent and lactoferrin.

In a further preferred form, the present invention provides a composition for inhibiting the colonisation and/or infection of mucous epithelium by bacteria, the composition including a mucolytic agent and lactoferrin.

In another preferred form, the present invention provides a composition for treating a disease or condition associated with bacterial infection of mucous epithelium, the composition including a mucolytic agent and lactoferrin.

In the case of a composition including lactoferrin for inhibiting and/or preventing colonisation and/or infection of mucous epithelium by Helicobacter pylori, or the treatment and/or prevention of a disease or condition associated with the infection of mucous epithelium by Helicobacter pylori, the mucolytic agent present in the composition is preferably N-acetyl cysteine.

The lactoferrin present in the composition may be lactoferrin further treated to improve its activity to inhibit colonisation or infection of mucous epithelium by bacteria. For example, the lactoferrin may be proteolytically digested to produce a protein fragment or polypeptide that displays improved activity with respect to inhibiting colonisation. To produce such a fragment, a protease such as gastric pepsin may be used to digest the lactoferrin under acidic conditions or by thermal inactivation at acidic pH by methods known in the art. Alternatively, a specific fragment of lactoferrin may be produced by recombinant means.

Accordingly, in a further preferred form, the present invention also provides a composition including a mucolytic agent and hydrolysed lactoferrin.

In a further preferred form, the present invention provides a composition for inhibiting the colonisation and/or infection of mucous epithelium by bacteria, the composition including a mucolytic agent and hydrolysed lactoferrin.

In another preferred form, the present invention provides a composition for treating a disease or condition associated with bacterial infection of mucous epithelium, the composition including a mucolytic agent and hydrolysed lactoferrin.

In the case of a composition including hydrolysed lactoferrin for inhibition and/or prevention of the colonization and/or infection of mucous epithelium by Helicobacter pylori, or the treatment and/or prevention of a disease or condition associated with the infection of mucous epithelium by Helicobacter pylori, the mucolytic agent present in the composition is preferably N-acetyl cysteine.

In another preferred form, the present invention provides a composition for inhibiting the colonisation and/or infection of mucous epithelium by bacteria, the composition including a mucolytic agent and one or more specific or cross-reactive antibodies to the bacteria colonising and/or infecting the mucous epithelium.

In a further preferred form, the present invention provides, a composition for treating a disease or condition associated with bacterial infection of mucous epithelium, the composition including a mucolytic agent and one or more specific or cross-reactive antibodies to the bacteria infecting the mucous epithelium.

In the case of a composition including one or more specific or cross-reactive antibodies for inhibition of the colonization and/or infection of mucous epithelium by H. pylori, or the treatment and/or prevention of a disease or condition associated with the infection of mucous epithelium by H. pylori, the mucolytic agent is preferably N-acetyl cysteine.

The present invention also provides a method for inhibiting bacterial colonisation of mucous epithelium in a biological system, the method including the step of administering to the biological system an effective amount of a mucolytic agent and egg or a component of egg that is capable of inhibiting bacterial colonisation in combination with the mucolytic agent.

Preferably, the egg is a hyperimmune egg resulting from the successive immunization of a bird with the bacteria (or antigens derived from the bacteria) for which colonisation or infection is to be inhibited, inflammation or damage associated with the bacterial infection is to be reduced, or the disease or condition associated with infection by the bacteria is to be treated.

For example, to inhibit the colonisation or infection by Helicobacter pylori in the gastrointestinal tract, hyperimmune egg from chickens inoculated with Helicobacter pylori may be used.

Accordingly, in a preferred from the present invention provides a method for inhibiting colonisation of the gastrointestinal tract by Helicobacter pylori, the method including the step of administering an effective amount of a mucolytic agent and hyperimmune egg or a component of hyperimmune egg.

The component of egg in the various forms of the present invention may be one or more components derived from egg that is capable of acting in combination with the mucolytic agent to inhibit colonisation or infection by the relevant bacteria, reduce inflammation or damage associated with infection by the relevant bacteria, or treat a disease or condition associated with infection by the relevant bacteria. As will be appreciated, such a component includes any fraction or extract derived from egg, including egg yolk. The component of egg may be produced by a method known in the art, including recombinant DNA technology, or any component derived from egg that is further treated or modified.

Preferably, the component of egg is egg yolk or one or more specific or cross-reactive antibodies to the bacteria from egg or egg yolk, including IgY antibodies.

Accordingly, in a preferred form the present invention provides a method for inhibiting bacterial colonisation of mucous epithelium in a biological system, the method including the step of administering to the biological system an effective amount of a mucolytic agent and one or more specific or cross-reactive IgY antibodies to the bacteria colonising the mucous epithelium.

For example, in the case of inhibiting the colonisation of the gastrointestinal tract by H. pylori, one or more specific IgY antibodies to H. pylori may be administered in combination with the mucolytic agent, or one or more IgY antibodies that cross-react with H. pylori may be administered in combination with the mucolytic agent.

In this regard, the one or more specific antibodies may be present in a mixture of other compounds, such as are present in egg yolk, or hyperimmune egg yolk. Alternatively, the antibodies may be in a substantially purified form, purified by a method known in the art, such as affinity purification of the antibodies from egg yolk, or hyperimmune egg yolk.

The amount of egg or a component of egg to be administered in the various forms of the present invention is also not particularly limited, so long as it is within such an amount, and in such a form, that generally exhibits a useful effect.

In this regard, a dose of the mucolytic agent and egg or a component of egg may be appropriately chosen, depending upon the particular mucolytic agent and the egg or component of egg used, the extent of bacterial colonisation or infection to be inhibited, the extent of inflammation or damage of mucous epithelium to be reduced, the tissue or organ colonised or infected, the kind of diseases or conditions to be treated, the age and body weight of the subject, the frequency of administration, or the presence of other active agents.

The mucolytic agent and egg or component of egg may be co-administered, or alternatively, be administered separately (so as to reach the desired site of action in combination) and either used alone or in conjunction with other agents to increase the likelihood of eradication of bacteria. Smaller doses may be applicable when used as an adjunctive therapy.

The administration of mucolytic agent and egg or a component of egg in the various forms of the present invention may be within any time suitable to produce the desired effect. For example, the administration may be prior to the onset of colonization, so as to prevent colonization. Alternatively, the administration may be during or after colonisation of mucous epithelium has occurred or been detected.

In another preferred form, the present invention provides a method for preventing bacterial colonisation of mucous epithelium in a biological system, the method including the step of administering to the biological system an effective amount of a mucolytic agent and egg or a component of egg that is capable of preventing bacterial colonisation in combination with the mucolytic agent.

In a human or animal system, the mucolytic agent and egg or a component of egg may be administered orally, or by any other suitable means, and therefore transit time of the mucolytic agent and egg or component of egg must be taken into account. Preferably, administration of the mucolytic agent and egg or a component of egg is by oral administration.

For oral administration of mucolytic agent and egg or a component of egg for inhibiting the colonisation or infection of the mucous epithelium of the gastrointestinal tract, preferably the mucolytic agent is N-acetyl cysteine and the egg is hyperimmune egg. For example, to inhibit the colonisation of the gastrointestinal tract with H. pylori, the mucolytic agent is preferably N-acetyl cysteine and the egg is hyperimmune egg from chickens immunised with H. pylori or antigens of H. pylori.

The administration of mucolytic agent and egg or a component of egg in the various forms of the present invention may also include the use of one or more acceptable additives, including acceptable salts, amino acids, polypeptides, polymers, solvents, buffers, excipients and bulking agents, taking into consideration the particular physical and chemical characteristics of the mucolytic agent and egg or a component of egg to be administered.

For example, the mucolytic agent and egg or a component of egg can be prepared into a variety of preparations in the form of, e.g., a food additive, an aqueous solution, an oily preparation, a fatty emulsion, an emulsion, a gel, etc., and these preparations can be administered orally, by adsorption or absorption, topically, rectally, nasally, bucally, or vaginally in dosage formulations containing conventional non-toxic acceptable carriers, or by any other convenient dosage form.

In the case of oral administration, the composition may be administered in the form of suitable oral preparations (for example solid preparations such as tablets, capsules, food additives, granules or powders; liquid or semi-liquid preparations such as egg yolk products, syrup, emulsions or suspensions).

Compositions containing the mucolytic agent and egg or a component of egg may also contain a preservative, stabiliser, dispersing agent, pH controller or isotonic agent. Examples of suitable preservatives are glycerol, propylene glycol, phenol or benzyl alcohol. Examples of suitable stabilisers are dextran, gelatin, α-tocopherol acetate or alpha-thioglycerin. Examples of suitable dispersing agents include polyoxyethylene (20), sorbitan mono-oleate (Tween 80), sorbitan sesquioleate (Span 30), polyoxyethylene (160) polyoxypropylene (30) glycol (Pluronic F68) or polyoxyethylene hydrogenated castor oil 60. Examples of suitable pH controllers include hydrochloric acid, sodium hydroxide and the like. Examples of suitable isotonic agents are glucose, D-sorbitol or D-mannitol.

The administration of the mucolytic agent and egg or a component of egg in the various forms of the present invention may also be in the form of a composition containing an acceptable carrier, diluent, excipient, suspending agent, lubricating agent, adjuvant, vehicle, delivery system, emulsifier, disintegrant, absorbent, preservative, surfactant, colorant, flavorant or sweetener, taking into account the physical and chemical properties of the particular mucolytic agent and egg or a component of egg used.

When administered orally, the composition will usually be formulated into unit dosage forms such as liquids, including long life liquid formulations for oral or topical administration, aqueous suspensions or solutions, tablets, cachets, powder, granules, beads, chewable lozenges, food additives, capsules, or similar dosage forms, using conventional equipment and techniques known in the art. Such formulations typically include a liquid, solid or semisolid carrier. Exemplary carriers include lactose, dextrose, sucrose, sorbitol, mannitol, starches, gum acacia, calcium phosphate, mineral oil, cocoa butter, oil of theobroma, alginates, tragacanth, gelatin, syrup, methyl cellulose, polyoxyethylene sorbitan monolaurate, methyl hydroxybenzoate, propyl hydroxybenzoate, talc, magnesium stearate, and the like.

In the case where the composition is administered as a tablet, the tablet may be made by compressing or moulding the active ingredient, with one or more accessory ingredients optionally included. Compressed tablets may be prepared by compressing, in a suitable machine, the active ingredient in a free-flowing form such as a powder or granules, optionally mixed with a binder, lubricant, inert diluent, surface active, or dispersing agent. Moulded tablets may be made in a suitable machine, by moulding together a mixture of the powdered active ingredient and a suitable carrier, moistened with an inert liquid diluent.

The carrier may also contain minor amounts of additives, such as substances that enhance solubility, isotonicity, and chemical stability, for example anti-oxidants, buffers and preservatives.

The administration of the mucolytic agent and egg or a component of egg in the various forms of the present invention may also utilize controlled release technology. The mucolytic agent and egg or component of egg may also be administered as a sustained-release product. To further increase the sustained release effect, the composition may be formulated with additional components such as vegetable oil (for example soybean oil, sesame oil, camellia oil, castor oil, peanut oil, rape seed oil); middle fatty acid triglycerides; fatty acid esters such as ethyl oleate; polysiloxane derivatives; alternatively, water-soluble high molecular weight compounds such as hyaluronic acid or salts thereof (weight average molecular weight: ca. 80,000 to 2,000,000), carboxymethylcellulose sodium (weight average molecular weight: ca. 20,000 to 400,000), hydroxypropylcellulose (viscosity in 2% aqueous solution: 3 to 4,000 cps), atherocollagen (weight average molecular weight: ca. 300,000), polyethylene glycol (weight average molecular weight: ca. 400 to 20,000), polyethylene oxide (weight average molecular weight: ca. 100,000 to 9,000,000), hydroxypropylmethylcellulose (viscosity in 1% aqueous solution: 4 to 100,000 cSt), methylcellulose (viscosity in 2% aqueous solution: 15 to 8,000 cSt), polyvinyl alcohol (viscosity: 2 to 100 cSt), polyvinylpyrrolidone (weight average molecular weight: 25,000 to 1,200,000).

The administration of the mucolytic agent and egg or a component of egg in the various forms of the present invention may further include the administration of other agents, including antibiotics and acid-suppressing agents.

Determination of the ability of a mucolytic agent and egg or a component of egg to inhibit colonisation of mucous epithelium may be confirmed by a suitable method known in the art. For example, the extent of inhibition of colonisation may be determined by directly visualizing the number of bacteria in a particular sample of mucous epithelium either with or without exposure to mucolytic agent and egg or a component of egg. In this case, the bacteria may be visualized by staining with a stain specific for detecting the bacteria of interest (for example, Giemsa staining of H. pylori), for example as described in “Saunders Dictionary and Encyclopedia of Laboratory Medicine and Technology” (Benington J. L).

Alternatively, bacterial colonisation may be determined by the extent of infiltration of the mucous sample by inflammatory cells in a particular sample of mucous epithelium with and without exposure to mucolytic agent and egg or a component of egg, for example as described in “Histology a Text and Atlas” (Third Edition; Ross, M. H., Rommell, L. J., Kaye, G. I. 1995 Williams and Wilkins Maryland, USA).

A further method for determining the ability of a mucolytic agent and egg or a component of egg to inhibit colonisation of mucous epithelium may be by the use of an enzyme marker such as myeloperoxidase as an index for neutrophil infiltration of the mucous epithelium, for example as described in Krawisz, J. et al. (1984) Gastroenterology 87:1344-1350. The samples of mucous epithelium for testing can be obtained by a suitable method known in the art, including biopsy of the mucous epithelium.

Preferably, the extent of inhibition of colonisation is such that the average number of bacteria per unit area of the mucous epithelium after treatment is reduced by 70% or more, as compared to the average number of bacteria per unit area in an untreated subject. More preferably, the extent of inhibition of colonisation is such that the average number of bacteria per unit area of the mucous epithelium after treatment is reduced by 85% or more, as compared to the average number of bacteria per unit area in an untreated subject. Most preferably, the extent of inhibition of colonisation is such that the average number of bacteria per unit area of the mucous epithelium after treatment is reduced by 90% or more, as compared to the average number of bacteria per unit area in an untreated subject.

The method of this form of the present invention may also include the administration of other agents, including antibiotics and acid-suppressing agents.

Preferably, the method of this form of the present invention also includes the administration of an antibiotic. An example of a suitable antibiotic is amoxicillin.

Accordingly, in another form the present invention provides a method for inhibiting bacterial colonisation of mucous epithelium in a biological system, the method including the step of administering to the biological system an effective amount of a mucolytic agent, an antibiotic and egg or a component of egg that that is capable of inhibiting bacterial colonisation in combination with the mucolytic agent.

In this regard, the method of this form of the present invention also reduces the likelihood of antibiotic resistance developing when antibiotic therapy is used.

Accordingly, the present invention also provides a method of reducing the development of antibiotic resistant bacteria in a biological system, the method including the step of administering to the biological system an effective amount of a mucolytic agent, and egg or a component of egg.

The present invention also provides a method for reducing bacterial infection of mucous epithelium in a biological system, the method including the step of administering to the biological system an effective amount of a mucolytic agent and egg or a component of egg that is capable of reducing bacterial infection in combination with the mucolytic agent.

Determination of the ability of a mucolytic agent and egg or a component of egg to reduce infection of mucous epithelium may be confirmed by a suitable method known in the art. For example, the extent of reduction of infection may be determined by directly visualizing the number of bacteria in a particular sample of mucous epithelium either with or without exposure to mucolytic agent and egg or a component of egg. In this case, the bacteria may be visualized by staining with a stain specific for detecting the bacteria of interest (for example, Giemsa staining of H. pylori) as described in “Saunders Dictionary and Encyclopedia of Laboratory Medicine and Technology” (Benington J. L).

Alternatively, bacterial infection may be determined by the extent of infiltration of the mucous sample by inflammatory cells in a particular sample of mucous epithelium with and without exposure to mucolytic agent and egg or a component of egg, for example as described in “Histology a Text and Atlas” (Third Edition; Ross, M. H., Rommell, L. J., Kaye, G. I. 1995 Williams and Wilkins Maryland, USA).

A further method for determining the ability of a mucolytic agent and egg or a component of egg to reduce infection of mucous epithelium may be by the use of an enzyme marker such as myeloperoxidase as an index for neutrophil infiltration of the mucous epithelium, for example as described in Krawisz, J. et al. (1984) Gastroenterology 87:1344-1350. The samples of mucous epithelium for testing can be obtained by a suitable method known in the art, including biopsy of the mucous epithelium.

Preferably, the extent of reduction of infection is such that the average number of bacteria per unit area of the mucous epithelium after treatment is reduced by 50% or more, as compared to the average number of bacteria per unit area in an untreated subject. More preferably, the extent of reduction of infection is such that the average number of bacteria per unit area of the mucous epithelium after treatment is reduced by 60% or more, as compared to the average number of bacteria per unit area in an untreated subject. Most preferably, the extent of reduction of infection is such that the average number of bacteria per unit area of the mucous epithelium after treatment is reduced by 80% or more, as compared to the average number of bacteria per unit area in an untreated subject.

In a preferred form the present invention provides a method for reducing bacterial infection of mucous epithelium in a biological system, the method including the step of administering to the biological system an effective amount of a mucolytic agent and one or more specific or cross-reactive IgY antibodies to the bacteria infecting the mucous epithelium.

As discussed previously, the administration of mucolytic agent and egg or a component of egg in the various forms of the present invention may be within any time suitable to produce the desired effect. For example, the administration may be prior to the onset of infection of the mucous epithelium. Alternatively, the administration may be during or after infection of the mucous epithelium has occurred or been detected.

In another preferred form, the present invention provides a method for preventing bacterial infection of mucous epithelium in a biological system, the method including the step of administering to the biological system an effective amount of a mucolytic agent and egg or a component of egg that is capable of preventing bacterial infection in combination with the mucolytic agent.

The method of this form of the present invention may also include the administration of other agents, including antibiotics and acid-suppressing agents.

Preferably, the method of this form of the present invention also includes the administration of an antibiotic. An example of a suitable antibiotic is amoxicillin.

Accordingly, in another form the present invention provides a method for reducing bacterial infection of mucous epithelium in a biological system, the method including the step of administering to the biological system an effective amount of a mucolytic agent, an antibiotic and egg or a component of egg that is capable of inhibiting bacterial infection in combination with the mucolytic agent.

The present invention provides a method for reducing inflammation of mucous epithelium associated with bacterial infection in a biological system, the method including the step of administering to the biological system an effective amount of a mucolytic agent and egg or a component of egg that is capable of reducing inflammation of mucous epithelium associated with bacterial infection in combination with the mucolytic agent.

Determination of the ability of a mucolytic agent and egg or a component of egg to reduce inflammation of mucous epithelium associated with bacterial infection may be confirmed by a suitable method known in the art. For example, the extent of inflammation may be determined by the extent of infiltration of the mucous sample by inflammatory cells in a particular sample of mucous epithelium with and without exposure to mucolytic agent and egg or a component of egg, as described in “Histology a Text and Atlas” (Third Edition; Ross, M. H., Rommell, L. J., Kaye, G. I. 1995 Williams and Wilkins Maryland, USA).

Alternatively, the extent of inflammation may be determined by use of an enzyme marker such as myeloperoxidase as an index for neutrophil infiltration of the mucous epithelium, for example as described in Krawisz, J. et al. (1984) Gastroenterology 87:1344-1350. The samples of mucous epithelium for testing can be obtained by a suitable method known in the art, including biopsy of the mucous epithelium.

Preferably, the extent of reduction of inflammation is such that the level of myeloperoxidase activity in a tissue sample of the mucous epithelium after treatment is reduced by 50% or more, as compared to the myeloperoxidase activity in a tissue sample from an untreated subject. More preferably, the extent of reduction of inflammation is such that the level of myeloperoxidase activity in a tissue sample of the mucous epithelium after treatment is reduced by 65% or more, as compared to the myeloperoxidase activity in a tissue sample from an untreated subject.

In another preferred form the present invention provides a method for reducing inflammation of mucous epithelium associated with bacterial in a biological system, the method including the step of administering to the biological system an effective amount of a mucolytic agent and one or more specific or cross-reactive IgY antibodies to the bacteria infecting the mucous epithelium.

As discussed previously, the administration of mucolytic agent and egg or a component of egg in the various forms of the present invention may be within any time suitable to produce the desired effect. For example, the administration may be prior to the onset of inflammation of mucous epithelium associated with bacterial infection of the mucous epithelium. Alternatively, the administration may be during or after the onset of inflammation of mucous epithelium associated with bacterial infection of the mucous epithelium has occurred or been detected.

In another preferred form, the present invention provides a method for preventing inflammation of mucous epithelium associated with bacterial infection in a biological system, the method including the step of administering to the biological system an effective amount of a mucolytic agent and egg or a component of egg that is capable of reducing inflammation of mucous epithelium associated with bacterial infection in combination with the mucolytic agent.

The method of this form of the present invention may also include the administration of other agents, including antibiotics and acid-suppressing agents.

Preferably, the method of this form of the present invention also includes the administration of an antibiotic. An example of a suitable antibiotic is amoxicillin.

Accordingly, in another form the present invention provides a method for reducing inflammation of mucous epithelium associated with bacterial infection in a biological system, the method including the step of administering to the biological system an effective amount of a mucolytic agent, an antibiotic and egg or a component of egg that is capable of reducing inflammation of mucous epithelium associated with bacterial infection in combination with the mucolytic agent.

In this regard, the method of this form of the present invention also reduces the likelihood of antibiotic resistance developing when antibiotic therapy is used.

The present invention provides a method for reducing damage to mucous epithelium associated with bacterial infection of the mucous epithelium in a biological system, the method including the step of administering to the biological system an effective amount of a mucolytic agent and egg or a component of egg that is capable of reducing the damage to mucous epithelium associated with bacterial infection in combination with the mucolytic agent.

Determination of the ability of a mucolytic agent and egg or a component of egg to reduce damage to mucous epithelium associated with bacterial infection may be confirmed by a suitable method known in the art, for example as described in “Histology a Text and Atlas” (Third Edition; Ross, M. H., Rommell, L. J., Kaye, G. I. 1995 Williams and Wilkins Maryland, USA). For example, the extent of damage may be determined by histopathological examination of tissue samples with and without exposure to mucolytic agent and egg or a component of egg.

In a preferred form the present invention provides a method for reducing damage to mucous epithelium associated with bacterial infection in a biological system, the method including the step of administering to the biological system an effective amount of a mucolytic agent and one or more specific or cross-reactive IgY antibodies to the bacteria infecting the mucous epithelium.

As discussed previously, the administration of mucolytic agent and egg or a component of egg in the various forms of the present invention may be within any time suitable to produce the desired effect. For example, the administration may be prior to the onset of damage to mucous epithelium associated with bacterial infection of the mucous epithelium. Alternatively, the administration may be during or after the onset of damage to mucous epithelium associated with bacterial infection of the mucous epithelium has occurred or been detected.

In another preferred form, the present invention provides a method for preventing damage to mucous epithelium associated with bacterial infection in a biological system, the method including the step of administering to the biological system an effective amount of a mucolytic agent and egg or a component of egg that is capable of reducing the damage to mucous epithelium associated with bacterial infection in combination with the mucolytic agent.

The method of this form of the present invention may also include the administration of other agents, including antibiotics and acid-suppressing agents.

Preferably, the method of this form of the present invention also includes the administration of an antibiotic. An example of a suitable antibiotic is amoxicillin.

Accordingly, in another form the present invention provides a method for reducing damage to mucous epithelium associated with bacterial infection of the mucous epithelium in a biological system, the method including the step of administering to the biological system an effective amount of a mucolytic agent, an antibiotic and egg or a component of egg that is capable of reducing damage to mucous epithelium associated with bacterial in combination with the mucolytic agent.

In this regard, the method of this form of the present invention also reduces the likelihood of antibiotic resistance developing when antibiotic therapy is used.

The present invention provides a method for treating a disease or condition associated with bacterial infection of mucous epithelium in a subject, the method including the step of administering to the subject an effective amount of a mucolytic agent and egg or a component of egg that is capable of treating the disease or condition associated with bacterial infection of mucous epithelium in combination with the mucolytic agent.

In another preferred form, the present invention provides a method for treating a disease or condition associated with bacterial infection of mucous epithelium in a subject, the method including the step of administering to the subject an effective amount of a mucolytic agent and one or more specific or cross-reactive IgY antibodies to the bacteria infecting the mucous epithelium.

As will be appreciated, the same considerations relevant to the administration of mucolytic agent and egg or a component of egg in regard to inhibiting bacterial colonisation or infection of mucous epithelium discussed above will also apply to the administration for treating a disease or condition associated with bacterial infection of mucous epithelium.

As discussed previously, the administration of mucolytic agent and egg or a component of egg in the various forms of the present invention may be within any time suitable to produce the desired effect. For example, the administration may be prior to the onset of a disease or condition in a subject associated with bacterial infection of the mucous epithelium. Alternatively, the administration may be during or after the onset of a disease or condition associated with bacterial infection of the mucous epithelium has occurred or been detected.

In another preferred form, the present invention provides a method for preventing a disease or condition associated with bacterial infection in a subject, the method including the step of administering to the subject an effective amount of a mucolytic agent and egg or a component of egg that is capable of preventing the disease or condition associated with bacterial infection of mucous epithelium in combination with the mucolytic agent.

The method of this form of the present invention may also include the administration of other agents, including antibiotics and acid-suppressing agents.

Preferably, the method of this form of the present invention also includes the administration of an antibiotic. An example of a suitable antibiotic is amoxicillin.

Accordingly, in another form the present invention provides a method for preventing and/or treating a disease or condition associated with bacterial infection of mucous epithelium in a subject, the method including the step of administering to the biological system an effective amount of a mucolytic agent, an antibiotic and egg or a component of egg that is capable of preventing and/or treating a disease associated with bacterial infection of mucous epithelium in combination with the mucolytic agent.

In this regard, the method of this form of the present invention also reduces the likelihood of antibiotic resistance developing when antibiotic therapy is used.

The present invention also provides the use of an effective amount of a mucolytic agent and egg or a component of egg for the preparation of a medicament for the treatment of a disease or condition associated with bacterial infection of mucdus epithelium.

The present invention also provides a composition including a mucolytic agent and egg or a component of egg.

The amount of the mucolytic agent to be used in the composition is not particularly limited, so long as it is within such an amount that generally will exhibit a therapeutic effect when the composition is administered to a subject.

The amount of egg or a component of egg to be used in the composition is also not particularly limited, so long as it is within such an amount that generally will exhibit an useful effect when the composition is administered to a subject.

In this regard, a dose of the mucolytic agent and egg or a component of egg used in the composition may be appropriately chosen, depending upon the particular mucolytic agent and egg or component of egg used, the extent of bacterial colonisation or infection to be inhibited, the extent of inflammation of mucous epithelium to be reduced, the tissue or organ colonised or infected, the kind of diseases or conditions to be treated, the age and body weight of the subject, the frequency of administration, and the presence of other active agents.

The mucolytic agent and egg or a component of egg may be co-administered in a single composition, or alternatively, be administered as separate compositions and thereby act at the desired site of action in combination.

In a preferred form, the present invention provides a composition for inhibiting the colonisation and/or infection of mucous epithelium by bacteria, the composition including a mucolytic agent and egg or a component of egg that is capable of preventing the colonisation and/or infection of mucous epithelium by bacteria.

In another preferred form, the present invention provides a composition for treating a disease or condition associated with bacterial infection of mucous epithelium, the composition including a mucolytic agent and egg or a component of egg that is capable of treating the disease associated with bacterial infection of mucous epithelium.

For a composition suitable for the administration of mucolytic agent and egg or a component of egg for inhibiting the colonisation or infection of the mucous epithelium of the gastrointestinal tract (or treating a disease or condition associated with bacterial infection of the gastrointestinal tract), preferably the mucolytic agent in the composition is N-acetyl cysteine and the egg is hyperimmune egg. For example, to inhibit the colonisation of the gastrointestinal tract with Helicobacter pylori, the composition preferably includes N-acetyl cysteine and hyperimmune egg or a component of hyperimmune egg from chickens immunised with H. pylori.

The composition may also include one or more acceptable additives, including salts, amino acids, polypeptides, polymers, solvents, buffers, excipients and bulking agents, taking into consideration the particular physical and chemical characteristics of the mucolytic agent and egg or a component of egg to be administered.

For example, the mucolytic agent and egg or a component of egg can be prepared into a variety of preparations in the form of, e.g., an aqueous solution, an oily preparation, a fatty emulsion, an emulsion, a gel, etc., and these preparations can be administered orally, by adsorption, absorption, topically, rectally, nasally, bucally, vaginally, or by any other convenient dosage form.

In the case of oral administration, the composition may be in the form of suitable oral preparations, including liquid preparations, syrup, emulsions or suspensions, or solid preparations such as tablets, capsules, granules or powders.

Compositions containing the mucolytic agent and egg or a component of egg may also contain a preservative, stabiliser, dispersing agent, pH controller or isotonic agent. Examples of suitable preservatives are glycerin, propylene glycol, phenol or benzyl alcohol. Examples of suitable stabilisers are dextran, gelatin, α-tocopherol acetate or alpha-thioglycerin. Examples of suitable dispersing agents include polyoxyethylene (20), sorbitan mono-oleate (Tween 80), sorbitan sesquioleate (Span 30), polyoxyethylene (160) polyoxypropylene (30) glycol (Pluronic F68) or polyoxyethylene hydrogenated castor oil 60. Examples of suitable pH controllers include hydrochloric acid, sodium hydroxide and the like. Examples of suitable isotonic agents are glucose, D-sorbitol or D-mannitol.

The composition may further include an acceptable carrier, diluent, excipient, suspending agent, lubricating agent, adjuvant, vehicle, delivery system, emulsifier, disintegrant, absorbent, preservative, surfactant, colorant, flavorant or sweetener, taking into account the physical and chemical properties of the particular mucolytic agent and the egg or a component of egg used.

When administered orally, the composition will usually be in a unit dosage form such as a liquid, including long life liquid formulations for oral or topical administration, aqueous suspensions or solutions, tablets, cachets, powder, granules, beads, chewable lozenges, food additives, capsules, or similar dosage forms, using conventional equipment and techniques known in the art. Such formulations typically include a solid, semisolid, or liquid carrier. Exemplary carriers include lactose, dextrose, sucrose, sorbitol, mannitol, starches, gum acacia, calcium phosphate, mineral oil, cocoa butter, oil of theobroma, alginates, tragacanth, gelatin, syrup, methyl cellulose, polyoxyethylene sorbitan monolaurate, methyl hydroxybenzoate, propyl hydroxybenzoate, talc, magnesium stearate, and the like.

In the case of administration involving a table, the tablet may be made by compressing or moulding the active ingredient, with one or more accessory ingredients as an option. Compressed tablets may be prepared by compressing, in a suitable machine, the active ingredient in a free-flowing form such as a powder or granules, optionally mixed with a binder, lubricant, inert diluent, surface active, or dispersing agent. Moulded tablets may be made by moulding in a suitable machine, a mixture of the powdered active ingredient and a suitable carrier moistened with an inert liquid diluent.

The carrier may-contain minor amounts of additives, such as substances that enhance solubility, isotonicity, and chemical stability, for example anti-oxidants, buffers and preservatives.

The composition may also include agents to allow controlled or sustained release of the mucolytic agent and egg or a component of egg. For example, in relation to a sustained release of the agents, the composition may be formulated with additional components such as vegetable oil (for example soybean oil, sesame oil, camellia oil, castor oil, peanut oil, rape seed oil); middle fatty acid triglycerides; fatty acid esters such as ethyl oleate; polysiloxane derivatives; alternatively, water-soluble high molecular weight compounds such as hyaluronic acid or salts thereof (weight average molecular weight: ca. 80,000 to 2,000,000), carboxymethylcellulose sodium (weight average molecular weight: ca. 20,000 to 400,000), hydroxypropylcellulose (viscosity in 2% aqueous solution: 3 to 4,000 cps), atherocollagen (weight average molecular weight: ca. 300,000), polyethylene glycol (weight average molecular weight: ca. 400 to 20,000), polyethylene oxide (weight average molecular weight: ca. 100,000 to 9,000,000), hydroxypropylmethylcellulose (viscosity in 1% aqueous solution: 4 to 100,000 cSt), methylcellulose (viscosity in 2% aqueous solution: 15 to 8,000 cSt), polyvinyl alcohol (viscosity: 2 to 100 cSt), polyvinylpyrrolidone (weight average molecular weight: 25,000 to 1,200,000).

Alternatively, the composition may have the mucolytic agent and egg or a component of egg incorporated into a hydrophobic polymer matrix for controlled release over a period of days. The composition may then be moulded into a solid form, or externally applied patch, suitable for providing efficacious concentrations of the mucolytic agent and egg or a component of egg over a prolonged period of time without the need for frequent re-dosing. Such controlled release films are well known to the art. Other examples of polymers commonly employed for this purpose that may be used include nondegradable ethylene-vinyl acetate copolymer a degradable lactic acid-glycolic acid copolymers which may be used externally or internally. Certain hydrogels such as poly(hydroxyethylmethacrylate) or poly(vinylalcohol) also may be useful, but for shorter release cycles than the other polymer release systems, such as those mentioned above.

The carrier may also be a solid biodegradable polymer or mixture of biodegradable polymers with appropriate time-release characteristics and release kinetics. The composition may then be moulded into a solid implant suitable for providing efficacious concentrations of the mucolytic agent and egg or a component of egg over a prolonged period of time without the need for frequent re-dosing. The mucolytic agent and egg or a component of egg can be incorporated into the biodegradable polymer or polymer mixture in any suitable manner known to one of ordinary skill in the art and may form a homogeneous matrix with the biodegradable polymer, or may be encapsulated in some way within the polymer, or may be moulded into a solid implant.

The composition according to the present invention may further include other agents, including antibiotics and acid-suppressing agents.

Preferably, the composition also includes the administration of an antibiotic. An example of a suitable antibiotic is amoxicillin.

In a preferred form, the present invention provides a composition for inhibiting the colonisation and/or infection of mucous epithelium by bacteria, the composition including a mucolytic agent and one or more specific or cross-reactive IgY antibodies to the bacteria colonising and/or infecting the mucous epithelium.

In a further preferred form, the present invention provides a composition for treating a disease or condition associated with bacterial infection of mucous epithelium, the composition including a mucolytic agent and one or more specific or cross-reactive IgY antibodies to the bacteria infecting the mucous epithelium.

In the case of a composition including one or more specific or cross-reactive IgY antibodies for inhibition and/or prevention of the colonization and/or infection of mucous epithelium by H. pylori, or the treatment and/or prevention of a disease or condition associated with the infection of mucous epithelium by H. pylori, the mucolytic agent in the composition is preferably N-acetyl cysteine.

### DESCRIPTION OF THE PREFERRED EMBODIMENTS

Reference will now be made to experiments that embody the above general principles of the present invention. However, it is to be understood that the following description is not to limit the generality of the above description.

### EXAMPLE 1

Model for Bacterial Colonisation

Female C57BL/6 mice, approximately six week old, were orally inoculated with Sydney strain 1 (SS1) of H. pylori (1.0×10⁸ bacteria delivered in 0.1 ml of 0.9% (w/v) sodium chloride) according to Lee et al., (1997) Gastroenterology 112:1386-1397 (the entire disclosure of which is incorporated herein by reference) and treated twice daily by oro-gastric gavage for 21 days, starting 4 weeks after inoculation (Scheme 1 below). Animals were housed in the Animal House at the Women's and Children's Hospital. All animals were fed mouse chow and water ad libitum.

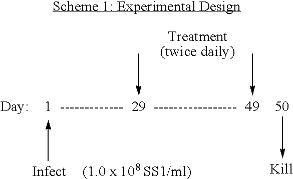

### EXAMPLE 2

Treatment

(a) Test Sample Preparation:

N-acetyl cysteine (NAC; Sigma Chemical Co., St Louis, Mo.) was dissolved in distilled water at 5% (w/v) and stored at 4° C. until use.

Non-immune bovine colostrum was prepared by pooled milkings. The freeze-dried powder was dissolved in distilled water at 20% (w/v) and stored at −20° C. prior to use.

Hyperimmune bovine colostrum (HBC; pooled milkings, freeze-dried powder) was dissolved in distilled water at 20% (w/v) and stored at −20° C. prior to use. Bovine lactoferrin (DMV International, Netherlands) was dissolved in distilled water at 3% (w/v) and stored at −20° C. until use.

Bovine lactoferrin hydrolysate-A (BLc-A) was prepared by proteolytic digestion of bovine lactoferrin with porcine gastric pepsin. The pH of the bovine lactoferrin solution was adjusted to pH 2.5 and porcine pepsin (Sigma) added at a final concentration of 3% (w/w of substrate). Hydrolysis was performed at 37° C. for 30 min and terminated by heating at 80° C. for 15 min,

Bovine lactoferrin hydrolysate-B (BLc-B) was prepared by thermal inactivation at pH 2.5 of bovine lactoferrin at 100° C. for 5 min.

Solutions of bovine hydrolysed lactoferrin were cooled to room temperature, adjusted to the original pH, filtered (0.45 μm) to remove any precipitation and stored at −20° C. until use.

(b) Treatment Regimen:

Treatment schedules are shown in Table 1.

TABLE 1
Treatment regimen

| | | | Treatment group: | | | | |
|---|---|---|---|---|---|---|---|
| | | | H₂O | BLf | BLc-A | BLc-B | HBC |
| | | | N= | | | | |
| | | | 9 | 10 | 11 | 11 | 11 |
| Time (daily): | | Tmt. | Dose (mL): | | | | |
| AM | 1 | H₂O | 0.1 | — | — | — | — |
| ″ | ″ | NAC | — | 0.1 | 0.1 | 0.1 | 0.1 |
| AM | 2 | H₂O | 0.1 | — | — | — | — |
| ″ | ″ | BLf | — | 0.1 | — | — | — |
| ″ | ″ | BLc-A | — | — | 0.1 | — | — |
| ″ | ″ | BLc-B | — | — | — | 0.1 | — |
| ″ | ″ | HBC | — | — | — | — | 0.1 |
| PM | 1 | H₂O | 0.1 | — | — | — | — |
| | ″ | NAC | — | 0.1 | 0.1 | 0.1 | 0.1 |
| PM | 2 | H₂O | 0.1 | — | — | — | — |
| ″ | ″ | BLf | — | 0.1 | — | — | — |
| ″ | ″ | BLc-A | — | — | 0.1 | — | — |
| ″ | ″ | BLc-B | — | — | — | 0.1 | — |
| ″ | ″ | HBC | — | — | — | — | 0.1 |

Triple therapy regimen comprised bismuth citrate, metronidazole and tetracycleine as decribed by Lee et al. (1997) Gastroenterology 112:1386-1397, except that bismuth citrate replaced bismuth SUBcitrate. Dose (mg/kg/day) was adjusted to deliver the same quantity as Bismuth/day.

### EXAMPLE 3

Assessment of Bacterial Colonisation and Pathology

Mice were killed by CO₂ asphyxiation followed by cervical dislocation and gastric tissue was collected for histological examination, bacterial culture and biochemical analysis as described previously in Lee et al. (1997) Gastroenterology 112:1386-1397 and also in Krawisz et al. (1984) Gastroenterology 87:1344-1350.

Briefly, the stomach was removed then cut along the greater curvature and rinsed in sterile saline to eliminate the stomach contents. Half of the tissue was fixed in 10% neutral buffered formalin, embedded in paraffin, cut into 5 μm sections and stained with hematoxylin and eosin (H & E) for histology and May-Grünwald-Giemsa (Giemsa) stain to assess bacterial colonisation. The remaining tissue was placed in 2 mL of sterile saline, weighed and homogenised for 30 seconds with an Ultra-Turrax homogeniser (Janke & Kenkel, Germany).

For bacterial culture, serial tenfold dilutions of tissue homogenate were performed and 200 μL of each dilution was plated out in duplicate on Helicobacter-selective agar (HSA) consisting of sterile lysed horse blood (5% v/v) in Columbia blood agar base (Oxoid Ltd., Basingstoke, England) containing Dent's supplement (Oxoid Ltd; 10 mg/L vancomycin, 5 mg/L trimethoprim, 5 mg/L cefsulodin and 5 mg/L amphotericin). Plates were incubated in a 10% CO₂ incubator set at 95% humidity at 37° C. for 5-7 days.

Myeloperoxidase (MPO) is an intracellular enzyme found in the granules of neutrophils and is therefore useful as an index of tissue neutrophil infiltration. The level of MPO was measured in 200 μL samples of homogenate. Suspensions were centrifuged at 15 000 g for 12 minutes in a Mikro benchtop centrifuge (Hettich, Germany) and the supematant discarded. The pellet was resuspended in 200 μL of hexadecyltrimethylammonium bromide (Sigma) detergent buffer (HTAB) then vortexed for 2 minutes and centrifuged at 15 000 g for 2 minutes. The supernatant was collected and 50 μL was added to 200 μL of reaction mixture (0.167 mg/L O-dianiside hydrochloride (Sigma), 0.05% hydrogen peroxide (30% w/v) and 10% phosphate buffer. Change in absorbance (OD min⁻¹) was measured at 450 nm using a Dynatech MR7000 spectrophotometer (Guernsey, Channel Islands) and MPO activity was calculated as: MPO (units) measured=OD min⁻¹/1.13×10⁻² where MPO unit=1 μmole H₂O₂ split, giving a change in OD=1.13×10⁻². MPO activity was expressed as MPO unit per gram of protein. Protein concentration in stomach tissue homogenate was determined using the Bio-Rad protein assay (Bio-Rad Laboratories Pty Ltd., Hercules, Calif.).

### EXAMPLE 4

Colonisation in Mice Treated with Hyperimmune Colostrum or Lactoferrin

The level of colonisation in the gastric body, transitional zone and antrum of mice inoculated with H. pylori was counted in 9-12 consecutive fields on Giemsa stained sections.

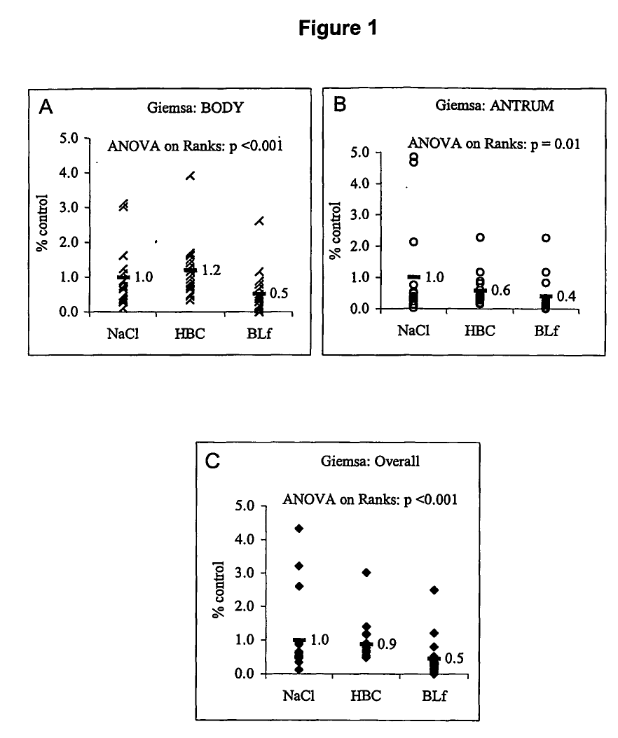

The level of colonisation in the gastric body of mice treated with saline (NaCl), hyperimmune bovine colostrum (HBC) or bovine lactoferrin (BLf) is shown in FIG. 1a. The level of colonisation in the antrum is shown in FIG. 1b. The level of colonisation in the mouse stomach when considered overall is shown in FIG. 1c.

As can be seen from the data, the level of colonisation in mice treated with hyperimmune colostrum or lactoferrin treatment was generally reduced as compared to the level of colonisation in mice treated with saline alone.

### EXAMPLE 5

Colonisation in Mice Treated with Lactoferrin with or without N-acetyl Cysteine

To assess the effect of the mucolytic agent N-acetyl cysteine in combination with bovine lactoferrin on colonisation, the level of colonisation in the gastric body, transitional zone and antrum in mice inoculated with H. pylori was counted in 9-12 consecutive fields on Giemsa stained sections.

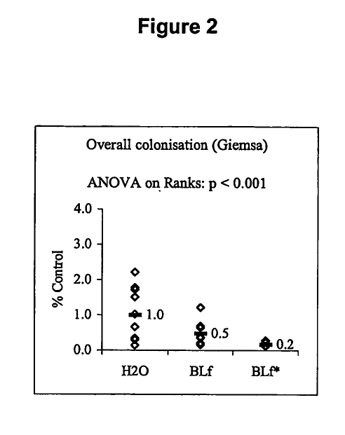

The overall level of colonisation in mice treated with water (H₂O), bovine lactoferrin (BLf), or bovine lactoferrin with N-acetyl cysteine (BLf*) is shown in FIG. 2.

As can be seen; the addition of the mucolytic agent N-acetyl cysteine to the treatment regime with bovine lactoferrin in mice significantly decreased the level of colonisation as compared to the treatment regime with bovine lactoferrin alone.

### EXAMPLE 6

Colonisation in Mice Treated with Non-Immune Colostrum, Hyperimmune Colostrum, Lactoferrin, or Lactoferin Hydrolysate, in Combination with N-acetyl Cysteine

The level of colonisation in the gastric body, transitional zone and antrum in mice inoculated with H. pylori was counted in 9-12 consecutive fields on Giemsa stained sections.

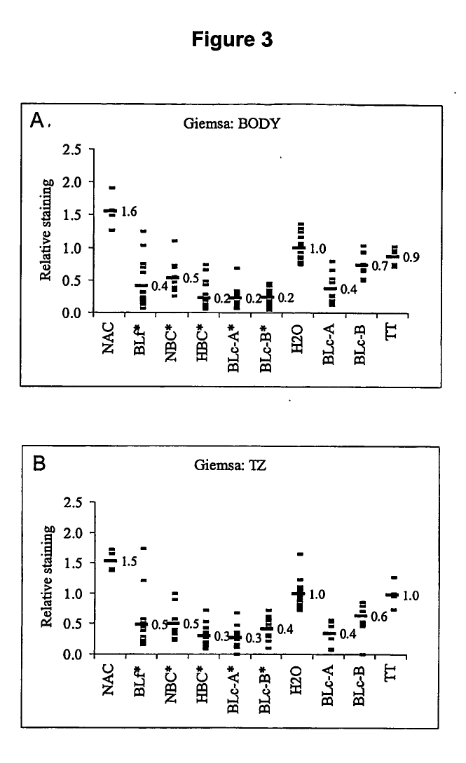

The level of colonisation in the gastric body of mice treated with water alone (H₂O), N-acetyl cysteine alone (NAC), bovine lactoferrin pepsin hydrolysate (BLc-A), bovine lactoferrin acid hydrolysate (BLc-B), non-immune bovine colostrum and N-acetyl cysteine (NBC*), hyperimmune bovine colostrum and N-acetyl cysteine (HBC*), bovine lactoferrin and N-acetyl cysteine (BLf*), bovine lactoferrin pepsin hydrolysate and N-acetyl cysteine (BLc-A*), bovine lactoferrin acid hydrolysate and N-acetyl cysteine (BLc-B*), or in mice treated with triple therapy regimen (TT) is shown in FIG. 3a.

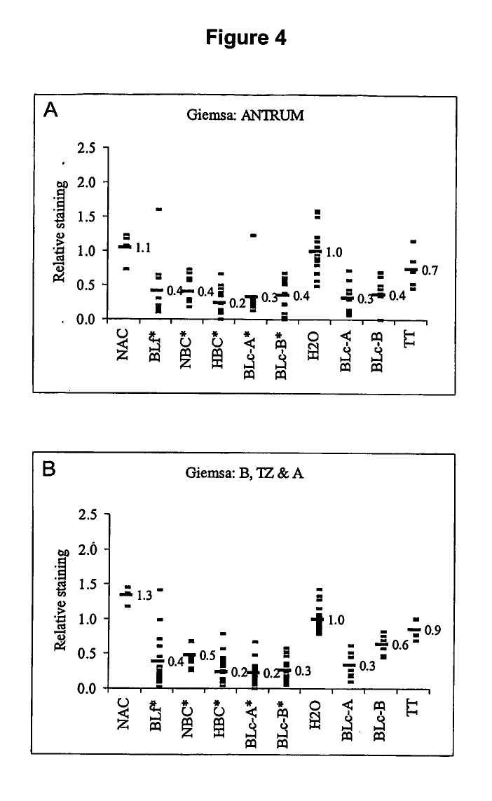

The level of colonisation in the transitional zone is shown in FIG. 3b. The level of colonisation in the antrum is shown in FIG. 4a. The level of colonisation in the mouse stomach when considered overall is shown in FIG. 4d.

As can be seen from the data, N-acetyl cysteine alone has no effect on the level of colonisation.

By comparison with the data shown in FIGS. 1a, 1b and 1c, it can be seen that N-acetyl cysteine improves the ability of colostrum to reduce the level of colonisation. The use of hyperimmune colostrum in combination with N-acetyl cysteine further improves the ability to reduce colonisation over non-immune colostrum.

As can also be seen, the use of N-acetyl cysteine improves the ability of lactoferrin hydroysate to reduce the level of colonisation (for both the pepsin derived hydrolysate and the acid derived hydrolysate) over the use of the hydrolysates alone.

### EXAMPLE 7

Effect of N-acetyl cysteine in combination with lactoferrin on inflammation

The level of inflammatory cell infiltration in mice inoculated with H. pylori was measured in the superficial, basal, submucosal, muscularis and serosal layers of the body, transitional zone and antrum in gastric tissue sections.

The sections were examined for structural changes to the stomach and scored on the presence (score: 1) or absence (score: 0) of normal architecture, lymphoid aggregates, cystic changes, loss of specialised cells and intestinal metaplasia.

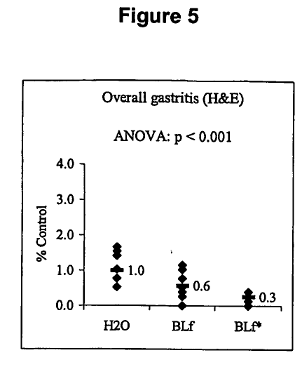

The overall level of chronic inflammatory cell infiltration (chronic gastritis) was determined in mice treated with water (H₂O), bovine lactoferrin (BLf), or bovine lactoferrin in combination with N-acetyl cysteine (BLf*). The data is shown in FIG. 5.

As can be seen, the presence of N-acetyl cysteine reduces the level of chronic inflammatory cell infiltration when used in combination with lactoferrin, over lactoferrin alone.

### EXAMPLE 8

Effect of N-acetyl Cysteine in Combination with Hyperimmune Colostrum, Lactoferin, or Lactoferrin Hydrolysate on Inflammation

The level of inflammatory cell infiltration in mice inoculated with H. pylori was measured in the superficial, basal, submucosal, muscularis and serosal layers of the body, transitional zone and antrum in gastric tissue sections.

The level of inflammatory cell infiltration was determined in mice treated with water alone (H₂O), hyperimmune bovine colostrum and N-acetyl cysteine (HBC), bovine lactoferrin and N-acetyl cysteine (BLf), bovine lactoferrin pepsin hydrolysate and N-acetyl cysteine (BLc-A), or bovine lactoferrin acid hydrolysate and N-acetyl cysteine (BLc-B).

The sections were examined for structural changes to the stomach and scored on the presence (score: 1) or absence (score: 0) of normal architecture, lymphoid aggregates, cystic changes, loss of specialised cells and intestinal metaplasia.

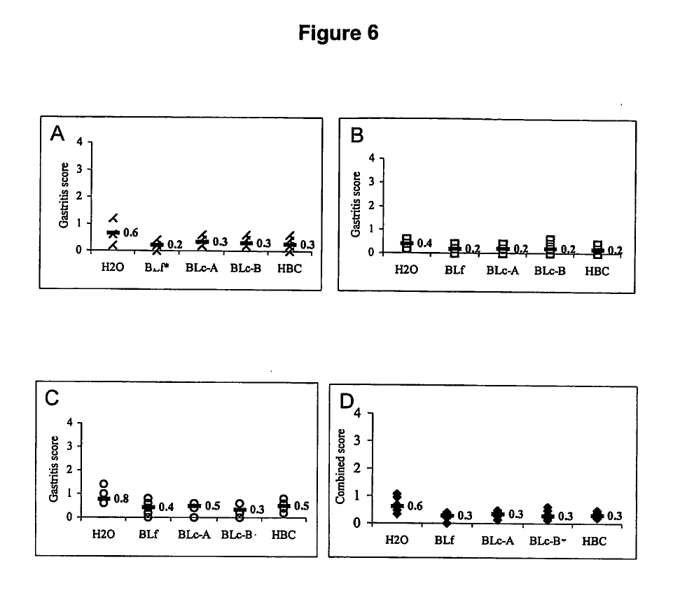

The data is shown in FIGS. 6a to 6d. As can be seen, the level of inflammation was reduced in all treatment groups as compared to the H₂O control group of mice. No inflammatory activity was observed in the other regions examined.

### EXAMPLE 9

MPO Activity in Mice Treated with Lactoferrin or Lactoferrin in Combination with N-acetyl Cysteine

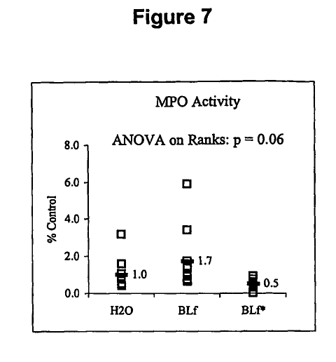

The level of acute gastritis (MPO activity) detected in mice treated with either bovine lactoferrin (BLf) or bovine lactoferrin in combination with N-acetyl cysteine (BLf*) was compared to the level of acute gastritis (MPO activity) in H₂O control mice. The data is shown in FIG. 7.

As can be seen, the extent of acute gastritis is reduced when N-acetyl cysteine is used in combination with lactoferrin, as compared to the use of lactoferrin alone.

### EXAMPLE 10

MPO Activity in Mice Treated with Hyperimmune Bovine Colostrum, Bovine Lactoferrin, or Bovine Lactoferrin Hydrolysate in Combination with N-acetyl Cysteine

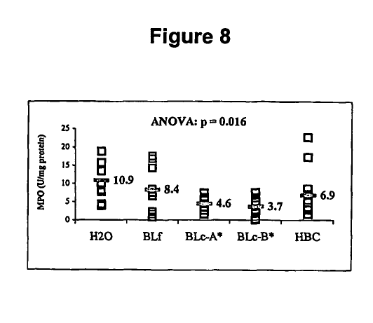

The level of acute gastritis (MPO activity) was determined in mice treated with water alone (H₂O), hyperimmune bovine colostrum and N-acetyl cysteine (HBC), bovine lactoferrin and N-acetyl cysteine (BLf), bovine lactoferrin pepsin hydrolysate and N-acetyl cysteine (BLc-A), or bovine lactoferrin acid hydrolysate and N-acetyl cysteine (BLc-B). The data is shown in FIG. 8.

As can be see, the extent of acute gastritis is reduced in all treatment groups as compared to the control.

### EXAMPLE 11

Comparison of the Effects of Hyperimmune Colostrum (HBC) and N-acetyl Cysteine (NAC)±Amoxicillin on H. pylori Colonisation and Associated Gastritis of the Mouse with Positive (Triple Therapy) and Negative (H2O) Treatment Groups

Female C57BL/6 mice, approximately six week old, were orally inoculated with Sydney strain 1 (SS1) of H. pylori (1.0×10⁸ bacteria delivered in 0.9% saline (0.1 ml) as described in Lee et al., (1997) Gastroenterology 112:1386-1397 and treated twice daily by oral-gastric gavage for 14 days. Animals were housed in the Animal House at the Women's and Children's Hospital. All animals were fed mouse chow and water ad libitum.

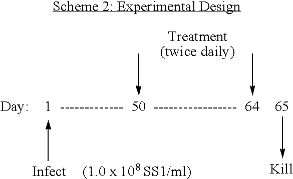

Test materials; N-acetyl cysteine, 50 mg/ml (NAC; Sigma Chemical Co., St Louis, Mo.) and omeprazole, 0.83 mg/ml (Losec®; Astra.Zeneca Pty Ltd, NSW, Australia) were both prepared fresh daily and stored at 4° C. until use. Hyperimmune bovine colostrum, 185 mg/ml (HBC: 02AO1, spray-dried powder), amoxycillin, 312.5 mg/ml (Amox; Alphapharm Pty Ltd, QLD, Australia) and metronidazole, 25 mg/ml (MTZ; Sigma) were stored at −20° C. until required. Amoxicillin was diluted 1/10 (in either HBC or MTZ solution) before use. All reagents were prepared in distilled water except Losec®, which was dissolved in 0.1 M sodium bicarbonate.

Treatment regimen;

Treatment are summarised in Table 2

TABLE 2

| | | Treatment group: | | | |
|---|---|---|---|---|---|
| Time | Galvage* | A (n = 18) | B (n = 18) | C (n = 18) | D (n = 18) |
| AM | 1 | H₂O | NAC | NAC | Losec |
| ″ | 2 | ″ | HBC | HBC/Amox | MTZ/Amox |
| PM | 1 | H₂O | NAC | NAC | Losec |
| ″ | 2 | ″ | HBC | HBC/Amox | MTZ/Amox |

*Mice were treated with two separate solutions (1 and 2) at every time point (AM and PM), each solution was delivered as 0.1 ml per mouse.

RESULTS: Viable Count:

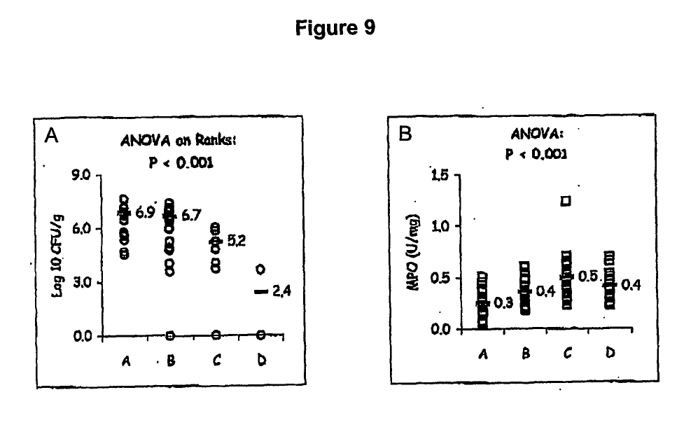

SS1 was recovered from 18/18 mice in the H₂O group, 17/18 mice treated with HBC and NAC, 9/18 of the mice treated with HBC, NAC and amoxycillin and only 1/18 of the mice treated with Losec®), metronidazole and amoxicillin. There was a significant difference in the numbers of viable bacteria recovered from the mice given H₂O and the mice treated either with HBC, NAC and amoxycillin or with Losec®, metronidazole and amoxicillin (p<0.05). Both of these groups were also significantly different from the animals treated only with HBC and NAC (p<0.05). No other differences were observed between the groups. The results are shown in FIG. 9, panel A.

Colonisation (Giemsa staining): The level of colonisation in the gastric body, transitional zone and antrum was counted in 9-12 consecutive fields on stained sections. In the body, the level of colonisation in mice treated with BLf, BLc-A or HBC was significantly different from the H₂O control group (p<0.05). Mice treated with HBC were also significantly different from mice treated with BLc-B (p<0.05). The same differences were observed in the transitional zone whereas in the antrum there was a significant difference between the BLf- and HBC-treated mice (but neither of the BLc-treated groups) and the control (p<0.05). When the results of the different sites were combined all of the treatment groups were different from the H₂O control (p<0.05).

Chronic Inflammation (H&E): Inflammatory cell infiltration was measured in the superficial, basal, submucosal, muscularis and serosal layers of the body, transitional zone and antrum in gastric tissue sections. There were some differences in the level of inflammation observed between treatment groups and the H₂O control group in the superficial and basal layers of the stomach mucosa. In the superficial layer, this was only significant (p<0.05) in mice treated with BLf±acid hydrolysis when the body, transitional zone and antrum scores were combined. In the basal layer the same result was obtained in the gastric body with BLf+acid hydrolysis but in this instance when the scores were combined the difference between the H₂O control and all of the treatment groups was significant (p<0.05). No inflammatory activity was observed in the other regions examined. When considered overall, in comparison with the control mice, the chronic inflammatory cell response was less severe in all of the treatment groups. This was significant (p<0.05) for all of the groups with the exception of mice treated with pepsin-digested BLf.

The sections were also examined for structural changes to the stomach and scored on the presence (score: 1) or absence (score: 0) of normal architecture, lymphoid aggregates, cystic changes, loss of specialised cells and intestinal metaplasia although no differences between the groups were observed.

Acute inflammation (MPO):

MPO activity (U/mg protein/min): The results are shown in FIG. 9, panel B. Briefly, the level of MPO detected in mice treated with either hydrolysate of BLf was approximately 60% less than that measured in the infected control mice treated with H;zO and this was significant (p<0.05). MPO measured was also 23% and 37% less in the groups treated with BLf and HBC respectively when compared with H₂ control mice but this was not statistically significant. Similarly, MPO detected in mice treated with hydrolysed BLf was approximately 40% lower than in HBC-treated mjce and 50% less than in BLf-treated mice but again this was not a significant difference. It should also be noted that the power of the experiment (with alpha: 0.050) was lower than the desired level of 0.800. Generally, the larger the sample size then tile greater the power of the test Therefore a larger group size than tested here was necessary to detect a statistical difference between treatment groups at the specified level of power and significance.

Finally, it will be appreciated that various modifications and variations of the methods and compositions of the invention described herein will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in the fields of the detection of chromosome abnormalities, prenatal diagnosis and preimplantation genetic diagnosis, molecular biology or related fields are intended to be within the scope of the present invention.

1. A method for inhibiting bacterial colonisation of mucous epithelium in a biological system, the method including the step of administering to the biological system an effective amount of a mucolytic agent and one or more of colostrum, hyperimmune milk, or a component of colostrum and/or hyperimmune milk that is capable of inhibiting bacterial colonisation in combination with the mucolytic agent.

2. A method according to claim 1, wherein the inhibition of bacterial colonisation occurs in the gastrointestinal tract.

3. A method according to claim 2, wherein the bacterial colonisation is colonisation by a Helicobacter species.

4. A method according to claim 3, wherein the Helicobacter species is Helicobacter pylori.

5. A method according to any one of claims 1 to 4, wherein the mucolytic agent is N-acetyl cysteine.

6. A method according to any one of claims 1 to 5, wherein the colostrum, hyperimmune milk, or a component of colostrum and/or hyperimmune milk are bovine colostrum, bovine hyperimmune milk, or a component of bovine colostrum and/or bovine hyperimmune milk.

7. A method according to any one of claims 1 to 6, wherein the colostrum is hyperimmune colostrum.

8. A method according to any one of claims 1 to 6, wherein the component of colostrum and/or hyperimmune milk is lactoferrin.

9. A method according to claim 8, wherein the lactoferrin is hydrolysed lactoferrin.

10. A method according to any one of claims 1 to 6, wherein the component of colostrum and/or hyperimmune milk is one or more specific or cross-reactive antibodies to the bacteria colonising the mucous epithelium.

11. A method according to any one of claims 1 to 10, wherein the method further includes the administration of an antibiotic.

12. A method according to claim 11, wherein the antibiotic is amoxycillin.

13. A method according to any one of claims 1 to 12, wherein the biological system is a human or animal.

14. A method for reducing bacterial infection of mucous epithelium in a biological system, the method including the step of administering to the biological system an effective amount of a mucolytic agent and one or more of colostrum, hyperimmune milk, or a component of colostrum and/or hyperimmune milk that is capable of reducing bacterial infection in combination with the mucolytic agent.

15. A method according to claim 14, wherein the reduction of bacterial infection occurs in the gastrointestinal tract.

16. A method according to claim 15, wherein the bacterial infection is infection by a Helicobacter species.

17. A method according to claim 16, wherein the Helicobacter species is Helicobacter pylori.

18. A method according to any one of claims 14 to 17, wherein the mucolytic agent is N-acetyl cysteine.

19. A method according to any one of claims 14 to 18, wherein the colostrum, hyperimmune milk, or a component of colostrum and/or hyperimmune milk are bovine colostrum, bovine hyperimmune milk, or a component of bovine colostrum and/or bovine hyperimmune milk.

20. A method according to any one of claims 14 to 19, wherein the colostrum is hyperimmune colostrum.

21. A method according to any one of claims 14 to 19, wherein the component of colostrum and/or hyperimmune milk is lactoferrin.

22. A method according to claim 21, wherein the lactoferrin is hydrolysed lactoferrin.

23. A method according to any one of claims 14 to 19, wherein the component of colostrum and/or hyperimmune milk is one or more specific or cross-reactive antibodies to the bacteria infecting the mucous epithelium.

24. A method according to any one of claims 14 to 23, wherein the method further includes the administration of an antibiotic.

25. A method according to claim 24, wherein the antibiotic is amoxycillin.

26. A method according to any one of claims 14 to 25, wherein the biological system is a human or animal.

27. A method for reducing damage to mucous epithelium associated with bacterial infection of the mucous epithelium in a biological system, the method including the step of administering to the biological system an effective amount of a mucolytic agent and one or more of colostrum, hyperimmune milk, or a component of colostrum and/or hyperimmune milk that is capable of reducing bacterial infection in combination with the mucolytic agent.

28. A method according to claim 27, wherein the damage occurs in the gastrointestinal tract.

29. A method according to claim 28, wherein the bacterial infection is infection by a Helicobacter species.

30. A method according to claim 29, wherein the Helicobacter species is Helicobacter pylori.

31. A method according to any one of claims 27 to 30, wherein the mucolytic agent is N-acetyl cysteine.

32. A method according to any one of claims 27 to 31, wherein the colostrum, hyperimmune milk, or a component of colostrum and/or hyperimmune milk are bovine colostrum, bovine hyperimmune milk, or a component of bovine colostrum and/or bovine hyperimmune milk.

33. A method according to any one of claims 27 to 32, wherein the colostrum is hyperimmune colostrum.

34. A method according to any one of claims 27 to 32, wherein the component of colostrum and/or hyperimmune milk is lactoferrin.

35. A method according to claim 34, wherein the lactoferrin is hydrolysed lactoferrin.

36. A method according to any one of claims 27 to 32, wherein the component of colostrum and/or hyperimmune milk is one or more specific or cross-reactive antibodies to the bacteria infecting the mucous epithelium.

37. A method according to any one of claims 27 to 36, wherein the method further includes the administration of an antibiotic.

38. A method according to claim 37, wherein the antibiotic is amoxycillin.

39. A method according to any one of claims 27 to 38, wherein the biological system is a human or animal.

40. A method for treating a disease or condition associated with bacterial infection of mucous epithelium in a subject, the method including the step of administering to the subject an effective amount of a mucolytic agent and one or more of colostrum, hyperimmune milk, or a component of colostrum and/or hyperimmune milk that is capable of treating the disease or condition associated with bacterial infection of mucous epithelium in combination with the mucolytic agent.

41. A method, according to claim 40, wherein the disease or condition is a disease or condition associated with bacterial infection of the gastrointestinal tract.

42. A method according to claim 41, wherein the disease or condition is gastric inflammation, an ulcer of the stomach or duodenum, non-ulcer dyspepsia, or a gastric condition associated with leukocyte infiltration.

43. A method according to any one of claims 40 to 42, wherein the bacterial infection is infection by a Helicobacter species.

44. A method according to claim 43, wherein the Helicobacter species is Helicobacter pylori.

45. A method according to any one of claims 40 to 44, wherein the mucolytic agent is N-acetyl cysteine.

46. A method according to any one of claims 40 to 45, wherein the colostrum, hyperimmune milk, or a component of colostrum and/or hyperimmune milk are bovine colostrum, bovine hyperimmune milk, or a component of bovine colostrum and/or bovine hyperimmune milk.

47. A method according to any one of claims 40 to 46, wherein the colostrum is hyperimmune colostrum.

48. A method according to any one of claims 40 to 46, wherein the component of colostrum and/or hyperimmune milk is lactoferrin.

49. A method according to claim 48, wherein the lactoferrin is hydrolysed lactoferrin.

50. A method according to any one of claims 40 to 46, wherein the component of colostrum and/or hyperimmune milk is one or more specific or cross-reactive antibodies to the bacteria infecting the mucous epithelium.

51. A method according to any one of claims 40 to 50, wherein the method further includes the administration of an antibiotic.

52. A method according to claim 51, wherein the antibiotic is amoxycillin.

53. A method according to any one of claims 27 to 38, wherein the subject is a human or animal.

54. A composition including a mucolytic agent and one or more of colostrum, hyperimmune milk, or a component of colostrum and/or hyperimmune milk.

55. A composition according to claim 54, wherein the mucolytic agent is N-acetyl cysteine.

56. A composition according to claims 54 or 55, wherein the colostrum, hyperimmune milk, or a component of colostrum and/or hyperimmune milk are bovine colostrum, bovine hyperimmune milk, or a component of bovine colostrum and/or bovine hyperimmune milk.

57. A composition according to any one of claims 54 to 56, wherein the colostrum is hyperimmune colostrum.

58. A composition according to any one of claims 54 to 56, wherein the component of colostrum and/or hyperimmune milk is lactoferrin.

59. A composition according to claim 58, wherein the lactoferrin is hydrolysed lactoferrin.

60. A composition according to any one of claims 54 to 56, wherein the component of colostrum and/or hyperimmune milk is one or more specific or cross-reactive antibodies to bacteria that colonise mucous epithelium.

61. A composition according to any one of claims 54 to 60, wherein the composition further includes an antibiotic.

62. A composition according to claim 61, wherein the antibiotic is amoxycillin.

63. A composition according to any one of claims 54 to 62, wherein the composition inhibits colonisation and/or infection of mucous epithelium by bacteria.

64. A composition according to claim 63, wherein the bacteria is a Helicobacter species.

65. A composition according to claim 64, wherein the Helicobacter species is Helicobacter pylori.
